(19) United States
(12) Patent Application Publication
(10) Pub. No.: US 2012/0029062 A1
(43) Pub. Date: Feb. 2, 2012

(54) Interior functionalized hyperbranched dendron-conjugated nanoparticles and uses thereof

(76) Inventors: Preethi H. Gunaratne, Houston, TX (US); Lalithya C. Jayarathne, Houston, TX (US); Matthew L. Anderson, Houston, TX (US)

(21) Appl. No.: 13/136,498

(22) Filed: Aug. 2, 2011

Related U.S. Application Data
(60) Provisional application No. 61/400,765, filed on Aug. 2, 2010.

(51) Int. Cl. A61K 31/7088; C12N 5/02; A61P 35/00; C07H 21/04; C07H 21/02; C07F 1/12; C12Q 1/02; A61K 31/28

(52) U.S. Cl. 514/44 R; 435/29; 435/375; 514/495; 536/23.1; 536/24.5; 556/28; 556/111

(57) ABSTRACT

Provided herein are nanoparticle platforms and combinatorial drug delivery vehicles comprising gold nanoparticles with a plurality of thilolated hyperbranched dendrons conjugated to the nanoparticle surface. The thiolated hyperbranched dendrons comprise chemically-modifiable surface groups, functionalized interior groups and nano-cavities within the hyperbranched structure to which a variety of payload molecules may be conjugated, optionally via a linker. Payload molecules may comprise nucleic acids, anticancer drugs and small molecule inhibitors, optionally with, non-cytotoxic signaling agents, for example, fluoroscein isothiocyanate. Also provided are methods for delivering one or more therapeutic agents to a cell or tissue or for treating a pathophysiological condition in a subject by delivering the combinatorial drug delivery vehicles to a cell or tissue associated with the pathophysiological condition to facilitate internalization of the vehicle to effect treatment.

### CROSS-REFERENCE TO RELATED APPLICATIONS

This non-provisional application claims benefit of priority under 35 U.S.C. §119(e) of provisional application U.S. Ser. No. 61/400,765, filed Aug. 2, 2010, now abandoned, the entirety of which is hereby incorporated by reference.

### GOVERNMENTAL SPONSORSHIP

The U.S. Government has a paid-up license in this invention and the rights in limited circumstances to require the patent owners to license others on reasonable terms as provided for by the terms of grant No. W81XWH-09 2-0139.

### BACKGROUND OF THE INVENTION

1. Field of the Invention

The present invention relates to the fields of nanoparticles, dendrons and cancer treatments. More specifically, the present invention relates to the design, synthesis, construction, characterization and use of gold nanoparticles modified by thiolated hyperbranched dendrons that facilitate the conjugation of different therapeutic agents thereto for delivery to various cancer and tumor cells.

2. Description of the Related Art

Gold nanoparticles (AuNPs) are currently being considered as vehicles for drug delivery, local tumor ablation and enhanced tumor detection. Gold nanoparticles are ideal carriers for therapeutic agents as their surfaces can be readily modified by thiolated hyperbranched dendrons with different interior groups, surface groups and nano-cavities to facilitate the conjugation of therapeutic agents of interest. Recently, thiolated siRNAs-conjugated AuNPs have been shown to be chemically stable and to be efficiently taken up by cells via an endocytic pathway. The key characteristics of nanoparticles that make them attractive delivery vehicles for therapeutic applications include the ability to manipulate size, payload density, duration of effect, and surface properties that can be further engineered for targeting purposes and for the selective delivery of therapeutic agents to disease cells and their release at the site of choice.

Materials with hydrodynamic diameters that are 10 nm or larger are spared from rapid kidney clearance and therefore can remain in the circulation for the duration of time necessary for long term action through slow release at low dosage. In addition, tumors have enhanced permeability and retention (EPR) characteristics and, at sizes of ˜100 nm, the tumors can selectively take up nanoparticles because they have poor lymphatic systems which cause the accumulation of these large molecules that then leak out of the vasculature into the tumor, i.e., extravasation. The extent to which nanoparticles can travel within the tumor following extravasation depends on size and surface charge. Particles of about 10 nm-100 nm in size with a small negatively charged surface are expected to be able to efficiently reach the tumor, be internalized and disseminated throughout the tumor following systemic administration.

Hyperbranched dendrimers have attracted attention as siRNA and drug delivery systems due to their large size, and enhanced permeability and retention characteristics. The dendrimer's interior branching groups, surface groups, nano-cavities and greater surface area per mass allows them to chemically conjugate or physically adsorb a large number of molecules. Both surface-functionalized AuNPs and hyperbranched dendrimers provide for the stable delivery of nucleic acids for treatment of disease with combinatorial drugs. In addition, bio-distribution studies show that dendrimers with higher molecular mass and more branches have longer circulation half-lives due to slower excretion into the urine.

However, there are limitations associated with the use of surface-functionalized AuNPs and hyperbranched dendrimers as delivery systems for therapeutic agents. For example, the dendrimer supported delivery systems have to overcome the possible leakage of nucleic acids or drugs, therefore compromising targeting of cancer cells and resultant selective elimination of disease cells. Moreover, the most commonly used generational dendrimer called polyamidoamine (PAMAM) with ethylenediamine (EDA) core is impure compared to PAMAM dendrimers with a diaminobutane (DAB) core according to the manufacturer. This is a problem as purity is the most important requirement in in vitro and in vivo applications as impurities that can accumulate in cells are cytotoxic. In addition to that, pure dendrimers provide more precise structures and higher number of interior branching groups, surface groups, and nano-cavities to carry higher number of nucleic acids or drugs.

The terminal amine groups in PAMAM dendrimer can become protonated giving a dendrimer a polycationic charge that can subsequently interact with serum albumin, the most abundant protein in plasma, thereby inhibiting specific targeting. Other limitations include the aggregation of PAMAM-interacted serum albumin in cells, leading to high cytotoxicity. Half generation anionic dendrimers have been shown to have high cellular uptake and generally less cytotoxic compared to full generation dendrimers. Neutral OH groups-terminated dendrimers which have low pKa values of interior tertiary amines (pKa=3-6) have been reported to be non-cytotoxic and prevent aggregation in cells.

The unique ability to silence patterns of gene expression underlying specific cellular contexts and disease conditions has earmarked microRNAs (miRNAs) as promising therapeutic agents in the context of personalized medicine. Because a single miRNA can potentially target and silence hundreds of genes across diverse signaling pathways, they offer powerful alternatives or complements to many of the small molecule inhibitors currently being developed, and obviate the need for high dose genotoxic chemotherapy. However, a major known obstacle to clinical application is the uncertainty on how best to identify and deliver miRNAs with maximal therapeutic impact. The main challenge to date is that miRNAs are unstable and degradable in the cellular environment. Furthermore, because of their widespread influence on patterns of gene expression there is a need to identify miRNAs that selectively affect disease cells and spare normal cells and to develop strategies that selectively deliver miRNAs to the aberrant cells that underlie disease conditions.

Therefore, the prior art is deficient in efficient, non-cytotoxic, non-viral delivery systems that can be multifunctionalized and easily delivered into living cells without the need for transfection reagents. Particularly, the prior art is deficient in interior functionalized hyperbranched dendron-conjugated gold nanoparticles (IFHD-AuNPs) designed to simultaneously carry a therapeutic nucleic acid and/or drugs, or a small molecule inhibitor, or any other agents of interest to cellular targets of interest in a selective manner. The present invention fulfills this longstanding need and desire in the art.

### SUMMARY OF THE INVENTION

The present invention is directed to a nanoparticle platform. The platform comprises a gold nanoparticle and a plurality of thiloated hyperbranched dendrons conjugated to the nanoparticle surface, said hyperbranched dendrons comprising chemically-modifiable surface groups, functionalized interior groups and nano-cavities within the hyperbranched structure. The present invention is directed to a related nanoparticle platform further comprising one or more of thiolated oligoethylene glycol, thiolated polyethylene glycol linkers or thiolated dendrons conjugated to uncovered nanoparticle surface areas or one or both of thiolated oligoethylene glycol or thiolated polyethylene glycol linkers conjugated to thiolated dendrons. The present invention is directed to another related nanoparticle platform further comprising one or more payload molecules conjugated to the interior or surface groups or within the nano-cavities or a combination thereof.

The present invention also is directed to a combinatorial drug delivery vehicle. The vehicle comprises a plurality of the nanoparticle platforms described herein and two or more different therapeutic agents conjugated to the surface and interior groups of the thiolated hyperbranched dendrons comprising the nanoparticle platforms. The present invention is directed to a related vehicle further comprising a non-cytotoxic signaling agent conjugated to the surface groups.

The present invention is directed further to another combinatorial drug delivery vehicle. The vehicle comprises a plurality of gold nanoparticles, a plurality of thilolated hyperbranched dendrons conjugated to the nanoparticle surface, said hyperbranched dendrons comprising chemically-modifiable tri(hydroxymethyl)amidomethane surface groups, a tertiary amine interior groups and nano-cavities within the hyperbranched structure, anticancer drugs and small molecule inhibitors individually conjugated to the tri(hydroxymethyl)amidomethane surface groups, where the surface groups or the anticancer drugs and small molecule inhibitors further comprise a chemical modifier, and microRNA duplexes electrostatically linked to the tertiary amine comprising the interior groups. The present invention is directed to a related vehicle further comprising fluorescein isothiocyanate conjugated to the tri(hydroxymethyl)amidomethane surface groups.

The present invention is directed further still to a method for delivering one or more therapeutic agents to a cell or tissue. The method comprises contacting the cell with the combinatorial drug delivery vehicle described herein. The payload molecules comprising the vehicle are therapeutic agents, such that the drug delivery vehicle is internalized into the cell, thereby delivering the one or more therapeutic agents thereto. In a related method the payload molecules further comprise a non-cytotoxic signaling agent and the method further comprises monitoring a signal from the signaling agent, thereby detecting the drug delivery vehicle in the cell.

The present invention is directed further still to a method for treating a pathophysiological condition in a subject. The method comprises administering, to the subject, an amount of the drug delivery vehicle described herein effective to deliver a pharmacological amount of payload molecules to cells or tissues associated with the pathophysiological condition, where the payload molecules comprise therapeutic agents, thereby treating the pathophysiological condition.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1G depict AuNP-miR-130b/miR-130b*-Cy3 synthesis, internalization and impact on target gene, GR-α. miRNA-conjugated AuNPs are rapidly internalized and released to influence patterns of gene expression. The scheme for synthesis of citrate-stabilized AuNPs is shown in FIG. 1A. The scheme for synthesis of miRNA duplexes-conjugated AuNPs from the citrate-stabilized AuNPs is shown in FIG. 1B. UV-visible absorption spectra of citrate-stabilized AuNPs before (left) and after (right) autoclaving are shown in FIG. 1C. Transmission electron microscopy (TEM) images of ˜13 nm citrate-stabilized AuNPs and AuNP-miR-130b/miR-130b*-Cy3 are shown in FIGS. 1D-1E, respectively. Three-dimensional confocal microscopy images of multiple myeloma (MM) cells (shown as a z-stack) 6 hrs after exposure to AuNPs and AuNP-miR-130b/miR-130b*-Cy3 are shown in the top and bottom rows respectively in FIG. 1F; top row are fluorescent image, bright field image and overlay of fluorescence and brightfield images of MM cells after exposure to citrate-stabilized AuNPs, while the bottom row are fluorescence image, bright field image and overlay of fluorescent and brightfield images of MM cells after exposure to AuNP-miR-130b/miR-130b*-Cy3, left to right in both rows. GR-α expression levels of MM cells treated with AuNP-miR-130b/130b*-Cy3 or miR-130b mimics (control) or non-treated for 6 hours are shown in FIG. 1G; AuNP-miR-130b/130b*-Cy3 are found to significantly reduce GR-α expression (p<0.01) compared to non-treated MM cells.

FIG. 2 demonstrates the cellular uptake of AuNP-miR-130b/miR-130b*-Cy3 by multiple myeloma (MM) cells lines, which have different sensitivity to drugs, called drug sensitive cells (MM.1S), drug resistant cells (MM.Re), and drug late resistant cells (MM.RL) via three-dimensional confocal microscopy. Images, shown as a z-stack, of these three different multiple myeloma (MM) cell lines are taken 20 min after exposure to AuNP-miR-130b/miR-130b*-Cy3 and are shown in three rows. The rows, from left to right, shows fluorescence image, bright field image and overlay of fluorescent and brightfield images of MM.1S cells (top), MM.Re cells (middle) and MM.RL cells (bottom).

FIGS. 3A-3B demonstrates that AuNP-miR-31/miR-31*Cy5 (FIG. 3A) inhibits cancer cell proliferation in ovarian OVCAR8 cancer cells compared to lentivirally delivered mir-31 (FIG. 3B).

FIGS. 4A-4B demonstrate that AuNP-miR-31/miR-31*Cy5 selectively kill p53-mutant ovarian cancer cells (OVCAR8 cells). p53 mutant OVCAR8 ovarian cancer cells (FIG. 4A) and p53 wild-type Hey ovarian cancer cells (FIG. 4B) are seeded into 96-well plates. After 24 hrs, culture media is replaced with OptiMEM media containing control or AuNP-miR-31/miR-31* at the concentrations shown. After 24 hrs, cell viability is measured using an MTS assay (Promega). Cell viability data (mean±s.e.m.) are normalized to OVCAR8 or HEY ovarian cancer cells treated with OptiMEM media only.

FIG. 5A is a schematic diagram of commercially available non-cytotoxic cystamine core PAMAM (generation 1.5 or G1.5) dendrimers with tertiary amine interior groups and tri(hydroxymethyl)amidomethane surface groups 1.

FIGS. 5B-5D are schematic diagrams of the internally quaternized tertiary amine interior groups in non-cytotoxic cystamine core PAMAM (generation 1.5 or G1.5) dendrimers with tri(hydroxymethyl)amidomethane surface group (2a) obtained from 1 that facilitate conjugation of negatively charged miRNA/miRNA*-S—S-poly(ethylene glycol) (PEG) duplexes. FITC-conjugated cystamine core PAMAM (generation 1.5 or G1.5) dendrimer (2b) and PEG-conjugated cystamine core PAMAM (generation 1.5 or G1.5) dendrimer (2c) are shown for comparison and clarity.

FIGS. 5E-5G are schematic diagrams showing the cleavage of non-cytotoxic internally quaternized cystamine core PAMAM (generation 1.5 or G1.5) dendrimers with tri(hydroxymethyl)amidomethane surface groups (2a) into two thiolated internally quaternized dendrons (3a; only one shown). FITC-conjugated thiolated dendrons (3b) and PEG-conjugated thiolated dendrons (3c) are shown for comparison and clarity.

FIG. 5H is a schematic diagram showing an interior functionalized hyperbranched dendron-conjugated nanoparticle (4), which includes internally quaternized thiolated dendrons (G1.5) with tri(hydroxymethyl)amidomethane surface groups (3a), FITC-conjugated thiolated dendrons (3b), and PEG-conjugated thiolated dendrons (3c) on to a Au NP surface to obtain IFHD-AuNPs.

FIGS. 5I-5J are schematic diagrams showing miRNA/miRNA*-S—S-poly(ethylene glycol) (PEG) duplexes (5a) or miRNA/miRNA*-Cy3 or Cy5 duplexes (5b) conjugated to interior functionalized hyperbranched dendron-conjugated nanoparticle (4).

FIGS. 5K-5L are schematic diagrams showing the construction of 7 via simultaneous conjugation of miRNA/miRNA*-S—S-PEG duplexes, miRNA/miRNA*-Cy3 or Cy5 duplexes cisplatin and small molecule inhibitors (MK-1775) to interior functionalized hyperbranched dendron-conjugated nanoparticles (6a,6b, 6c).

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The terms “comprise” and “comprising” are used in the inclusive, open sense, meaning that additional elements may be included.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the terms “subject” or “individual” refers to a mammal, preferably a human, who is a recipient of any therapeutic agent or other molecule as delivered via the gold nanoparticle platforms or combinatorial drug delivery vehicles described herein.

In one embodiment of the present invention there is provided a nanoparticle platform, comprising a gold nanoparticle; and a plurality of thiloated hyperbranched dendrons conjugated to the nanoparticle surface, said hyperbranched dendrons comprising chemically-modifiable surface groups, functionalized interior groups and nano-cavities within the hyperbranched structure.

Further to this embodiment the nanoparticle platform comprises one or more of thiolated oligoethylene glycol, thiolated polyethylene glycol linkers or thiolated dendrons conjugated to uncovered nanoparticle surface areas or one or both of thiolated oligoethylene glycol or thiolated polyethylene glycol linkers conjugated to thiolated dendrons. In another further embodiment, the nanoparticle platform comprises one or more payload molecules conjugated to the interior or surface groups or within the nano-cavities or a combination thereof. In this further embodiment the payload molecules may be one or more therapeutic agents or a non-cytotoxic signaling agent or a combination thereof. Particularly, the therapeutic agents may comprise one or more nucleic acids, one or more anticancer drugs, one or more small molecule inhibitors, or a combination thereof. For example, the nucleic acid may be a microRNA, a small-interfering RNA or a DNA, the anticancer drug may be cisplatin, the small molecule inhibitor may be MK-1775, and the non-cytoxic signaling agent may be fluoroscein isothiocyanate.

In one aspect of this further embodiment the nucleic acid further may comprise a thiolated polyethylene glycol linker or a thiolated oligoethylene glycol linker conjugated to an antisense strand of the nucleic acid. In another aspect, the therapeutic agent may be one or more nucleic acids where the nucleic acids are electrostatically linked to the functionalized interior groups. In yet another aspect the therapeutic agent may be one or more anticancer drugs or one or more small molecule inhibitors or a combination thereof where the agent(s) are conjugated to chemically-modified surface groups or where the agent(s) comprise a chemical modification and are conjugated directly to unmodified surface groups.

In all embodiments and aspects the surface groups may comprise tri(hydroxymethyl)amidomethane and the interior groups may comprise a tertiary amine. Also in all embodiments the surface groups may be chemically modified with an amino, sodium craboxylate, amido ethanol, succinamic acid, hexylamine, or amidoethylethanolamine moiety.

In another embodiment of the present invention, there is provided a combinatorial drug delivery vehicle, comprising a plurality of the nanoparticle platforms as described supra; and two or more different therapeutic agents conjugated to the surface and interior groups of the thiolated hyperbranched dendrons comprising the nanoparticle platforms. In a further embodiment the combinatorial drug delivery vehicle comprises a non-cytotoxic signaling agent conjugated to the surface groups. An example of the non-cytotoxic signaling molecule is fluorescein isothiocyanate.

In a related embodiment of the present invention there is provided a combinatorial drug delivery vehicle, comprising a plurality of gold nanoparticles; a plurality of thilolated hyperbranched dendrons conjugated to the nanoparticle surface, said hyperbranched dendrons comprising chemically-modifiable tri(hydroxymethyl)amidomethane surface groups, a tertiary amine interior groups and nano-cavities within the hyperbranched structure; anticancer drugs and small molecule inhibitors individually conjugated to the tri(hydroxymethyl)amidomethane surface groups, where either the surface groups or the anticancer drugs and small molecule inhibitors further comprise a chemical modifier; and microRNA duplexes electrostatically linked to the tertiary amine comprising the interior groups.

In a further embodiment, the combinatorial drug delivery vehicle further comprises fluorescein isothiocyanate conjugated to the tri(hydroxymethyl)amidomethane surface groups. In both embodiments one or both of the unconjugated tri(hydroxymethyl)amidomethane surface groups or an antisense strand of the microRNA duplex further comprises a thiolated polyethylene glycol linker or a thiolated oligoethylene glycol linker. Also, the chemical modifier is an amino, sodium craboxylate, amido ethanol, succinamic acid, hexylamide, or amidoethylethanolamine moiety. In addition, the anticancer drug is cisplating and the small molecule inhibitor is MK-1775.

In yet another embodiment of the present invention, there is provided a method for delivering one or more therapeutic agents to a cell or tissue, comprising contacting the cell with the drug delivery vehicle of as described supra, where the payload molecules comprise therapeutic agents, such that the drug delivery vehicle is internalized into the cell, thereby delivering the one or more therapeutic agents thereto. In a further embodiment the payload molecules further comprise a non-cytotoxic signaling agent, the method comprising monitoring a signal from the signaling agent, thereby detecting the drug delivery vehicle in the cell. An example of the non-cytotoxic signaling molecule is fluorescein isothiocyanate. In both embodiments the therapeutic agents may be a microRNA, cisplatin and MK-1775. Also in both embodiments the contacting step may occur in vitro or in vivo.

In yet another embodiment of the present invention, there is provided a method for treating a pathophysiological condition in a subject, comprising administering, to the subject, an amount of the combinatorial drug delivery vehicle as described supra effective to deliver a pharmacological amount of payload molecules to cells or tissues associated with the pathophysiological condition, where the payload molecules comprise therapeutic agents, thereby treating the pathophysiological condition. The therapeutic agents may be as described supra. Also, the pathophysiological condition may be a cancer.

Provided herein are thiolated hyperbranched dendron modified gold nanoparticle compositions or nanoconjugates, systems and methods useful as platforms for delivery of various payloads, such as therapeutic agents, to cells, tumors, or tissues of interest. Particularly, the present invention provides for the design, synthesis, construction, characterization, and use of interior functionalized hyperbranched dendron-conjugated gold nanoparticles (IFHD-AuNPs) that can conjugate and deliver therapeutic agents to various cancer and tumor cells to 1) selectively inhibit gene expression, 2) act on target mRNAs and arrest the protein synthesis, 3) eradicate already created cancer and premalignant cells, 4) suppress the tumor and prevent recurrence of the diseases, and 5) sensitize cancer cells to existing therapies including as chemotherapy, small molecule inhibitors, radiation, hyperthermal therapy. More particularly, gold (Au) nanoparticles (NPs) have surfaces that can be modified readily with thiolated hyperbranched dendrons that comprise functionalized interior groups, surface groups, and nano-cavities effective to facilitate conjugation of payload molecules thereto. These interior functionalized hyperbranched dendron-conjugated gold nanoparticles (IFHD-AuNPs) are efficiently internalized in cells, which impacts biologic activity, phenotype, and patterns of gene expression in a sequence specific manner.

The dendrons disclosed herein provide a higher number of surface groups, and a greater number of interior branching groups, surface groups and nano-cavities compared to dendrimers with an EDA core, which causes cell cytotoxicity. More importantly, the dendrons described herein are readily available with different interior branching groups, surface groups, and nano-cavities and are therefore able to conjugate to various therapeutic agents of interest depending on their functionality to the dendron's interior groups or surface groups or nano-cavities. The dendrons may comprise a G1.5, G2.5, G3.5, G4.5, G5.5 or higher half generation. For example, the hyperbranched dendrons described herein are suitable to facilitate conjugation of one or more different nucleic acids to interior groups and/or conjugation of one or more different anticancer drugs and one or more small molecule inhibitors to functionalized surface groups and, as such, to deliver the one or more therapeutic agents or other molecule(s) in a single delivery system. Alternatively, the anticancer drug(s) and/or small molecule inhibitor(s) are modified or functionalized and are conjugated directly to the dendrons' surface groups.

A payload may comprise therapeutic agents and/or other molecules, such as a non-toxic signaling agent, e.g., fluorescein isothiocyanate or other nontoxic fluorophore or dye for the purposes of tracking the bio distribution of the conjugates. Also, a signaling agent comprising the drug delivery vehicles are useful for locating tumors, cancer cells and cancer metastases. Therapeutic agents useful as conjugates to the IFHD-AuNPs described herein may be any agent, such as, but not limited to, nucleic acids, anticancer drugs and small molecule inhibitors. Particularly, nucleic acids useful in the invention are microRNAs, siRNAs and DNAs. Anticancer drugs are well-known in the art and therapeutic efficacy may be dependent on the type of cancer or tumor. In a non-limiting example, cisplatin is an anticancer drug that readily conjugates to the IFHD-AuNPs. A non-limiting example of a small molecule inhibitor is MK-1775, 2-allyl-1-(6-(2-hydroxypropan-2-yl)pyridin-2-yl)-6-((4-(4-methylpiperazin-1-yl)phenyl)amino)-1H-pyrazolo[3,4-d]pyrimidin-3(2H)-one.

Particularly, the IFHD-AuNP nanoconjugates described herein are useful in enabling an increase in the payload density of the therapeutic agents and/or other molecules in a single IFHD-AuNP delivery system thereby providing simultaneous delivery of all conjugated payload agents or molecules at the site of interest. For example, surfactants, such as, but not limited to, thiolated oligoethylene glycol or thiolated polyethylene glycol, may be conjugated directly to uncovered surface areas of the nanoparticle surface or indirectly via conjugation to dendrons or via conjugation to therapeutic agents, for example, nucleic acids. This effectively increases or decreases the density of the therapeutic agents or other molecules.

The IFHD-Au NP delivery system provides a combinatorial strategy that incorporates different therapeutic agents, e.g., miRNAs with small molecule inhibitors and anticancer drugs or chemotherapeutic agents, into the same drug delivery system in a precisely controllable manner. In distinct contrast to systems requiring payloads of the same types of therapeutic agents, this combinatorial strategy enables the loading of multiple therapeutic agents onto the same delivery system with a predefined stoichiometric ratio. The functionalized interior groups or surface groups function as cleavable linkers between the dendrons and the therapeutic agents. Thus, the therapeutic activity of the individual agents is activated or enabled after the therapeutic conjugates are delivered into the target cells and unloaded from the delivery system. For example, once internalized miRNAs are successfully released from the IFHD-AuNPs to influence patterns of gene expression. Moreover, the bioconjugated miRNAs significantly impacted patterns of gene expression and inhibited cell proliferation in vitro. The combinatorial strategy of IFHD-AuNPs to carry both miRNAs and anticancer drugs can significantly sensitize tumors or cancer cells to the anticancer drug.

Moreover, the non-cytotoxic interior functionalized hyperbranched dendron-conjugated gold nanoparticle (IFHD-Au NP) system is an appropriate delivery system to overcome the current limitations encountered with known delivery systems. The hierarchical architecture of dendrons enables the attachment of larger number of therapeutic agents or any other molecule of interest and has low cytotoxicity compared to PAMAM dendrimers, as IFHD AuNPs do not contain EDA cores. Furthermore, IFHD AuNPs enhance the permeability and retention effect in the blood stream compared to dendrimers. For example one ˜13 nm Au NP can be modified with larger numbers of dendrons compared to the number of dendrons in a dendrimer per se. Therefore, a possible leakage of one to all upon dilution in the circulatory system can be prevented using the IFHD-AuNPs compared to dendimer itself because thiolated dendrons-conjugated AuNPs form a stable system which can reach a target without loss of the payload.

Dendrons can be labeled using non-cytotoxic signaling agents, such as fluorescein isothiocyanate (FITC), to assay the internalization of therapeutic agents-conjugated IFHD-AuNPs into cells. Consequently, IFHD-AuNP delivery system provide an alternative to using cytotoxic Cy3 or Cy5 antisense strand-labeled miRNA duplexes. This is an important improvement over known systems, as Cy3 or Cy5 antisense strand-labeled duplexes are known in the art to be very expensive and to accumulate in the cells, which in turn create high cytotoxicities, an undesirable attribute.

Thus, the present invention provides methods of delivering a combination of therapeutic agents to a cell or tissue, whether the cell or tissue is healthy or in a diseased state. Contacting the cells or tissue with the drug delivery vehicles described herein results in internalization of the vehicle. Contacting the cells or tissue in vitro or ex vivo may utilize any standard or well-known method that brings the drug delivery vehicles into contact with the cell or tissue such that internalization of the vehicles is facilitated. In vitro or ex vivo this is achieved by exposing the cells or tissue to the drug delivery vehicle in a suitable medium. For in vivo applications, any known method of administration is suitable as described herein. Without being limiting administration may be orally, intranasally or through intravenous (IV), intramuscular (IM) or intraperioneal (IP) injection.

As such, also provided are methods for treating a pathophysiological condition, for example, but not limited to, a cancer, using the IFHD-AuNP nanoconjugates or delivery system. One of ordinary skill in the art is well able to determine effective combinations of therapeutic agents and whether or not a signaling agent should be incorporated into the nanoparticle composition. Moreover one of ordinary skill in the art is well able to determine an appropriate amount of the IFHD-AuNP nanoconjugates effective to deliver a pharmacologically effective amount of the therapeutic agents to a subject. Dosage determinations are routinely based on, but not limited to, the therapeutic agents used, the age and sex of the subject, the overall health of the subject, the type of cancer, and the remission or progression of the cancer. Moreover, one of ordinarly skill in the art is well able to determine or measure a result or therapeutic effect of the agents comprising the IFHD-AuNP or to detect and quantify the signal produced by a signaling agent comprising the IFHD-AuNP upon internalization into a cell, whether in vitro, in vivo or ex vivo, using known and standard methodologies.

While the invention described here exemplifies the design and construction of a novel drug delivery vehicle, composition or system designed to conjugate microRNA, cisplatin, MK-1775 and fluorocein isocyanate to gold nanoparticles via thiolated hyperbranched dendrons, one of ordinary skills in the art, with the benefit of this disclosure, is well suited to extend the disclosed design to other suitable nanomaterials, functional groups and/or surfactants, etc. to facilitate conjugation of any therapeutic agent.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### AuNP-miR-130b/miR-130b*-Cy3 Nanoparticles

### Synthesis

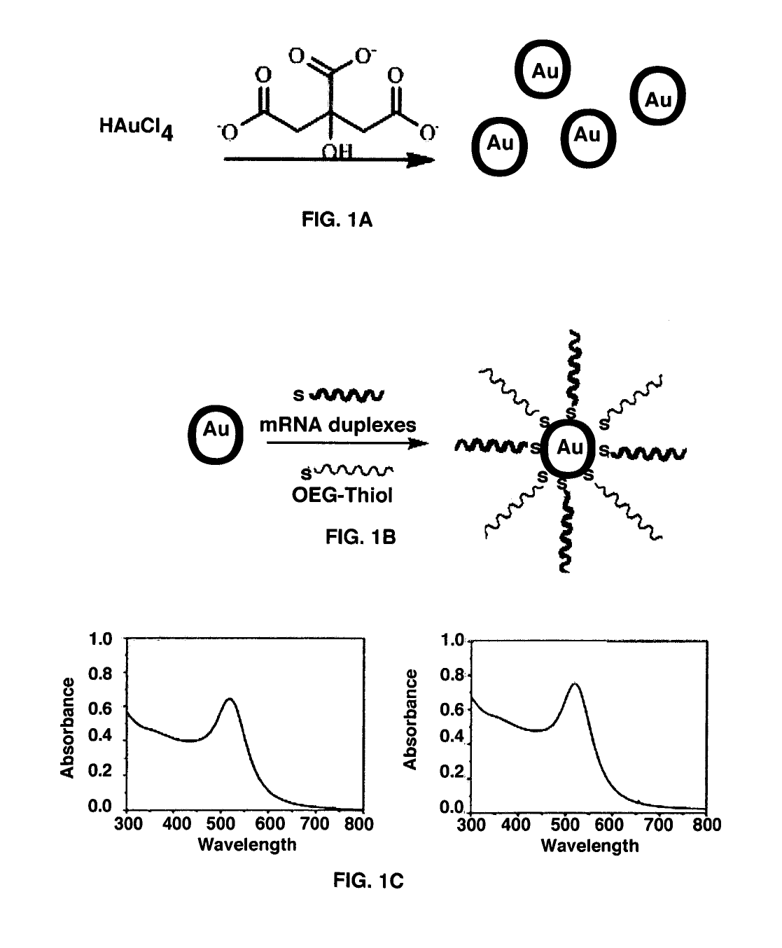

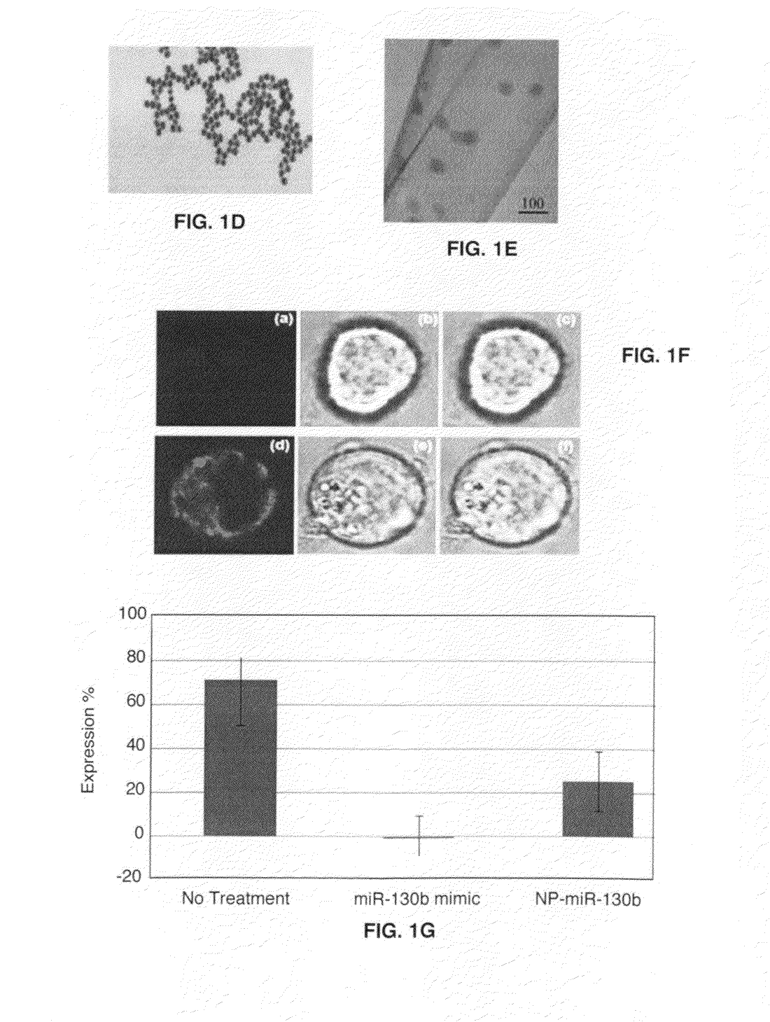

AuNP-miR-130b/miR-130b*-Cy3 are synthesized and their internalization and impact on target gene GR-α are measured. FIG. 1 shows that miRNA-conjugated AuNPs are rapidly internalized and released to influence patterns of gene expression. The scheme for synthesis of citrate-stabilized AuNPs is shown in FIG. 1A. The scheme for synthesis of miRNA duplexes-conjugated AuNPs from the citrate-stabilized AuNPs is shown in FIG. 1B. UV-visible absorption spectra of citrate-stabilized AuNPs before and after autoclaving are shown in FIG. 1C. Transmission electron microscopy (TEM) images of ˜13 nm citrate-stabilized AuNPs and AuNP-miR-130b/miR-130b*-Cy3 are shown in FIG. 1D and FIG. 1E, respectively. Three-dimensional confocal microscopy images of multiple myeloma (MM) cells (shown as a z-stack) 6 hrs after exposure to AuNPs and AuNP-miR-130b/miR-130b*-Cy3 are shown in the top and bottom rows respectively in FIG. 1F where the top row, left to right, are fluorescent image, bright field image and overlay of fluorescence and brightfield images of MM cells after exposure to citrate-stabilized AuNPs, while the bottom row, left to right, are fluorescence image, bright field image and overlay of fluorescent and brightfield images of MM cells after exposure to AuNP-miR-130b/miR-130b*-Cy3. GR-α expression levels of MM cells treated with AuNP-miR-130b/130b*-Cy3 or miR-130b mimics (control) or non-treated for 6 hours are shown in FIG. 1G; AuNP-miR-130b/130b*-Cy3 are found to significantly reduce GR-α expression (p<0.01) compared to non-treated MM cells.

### Internalization

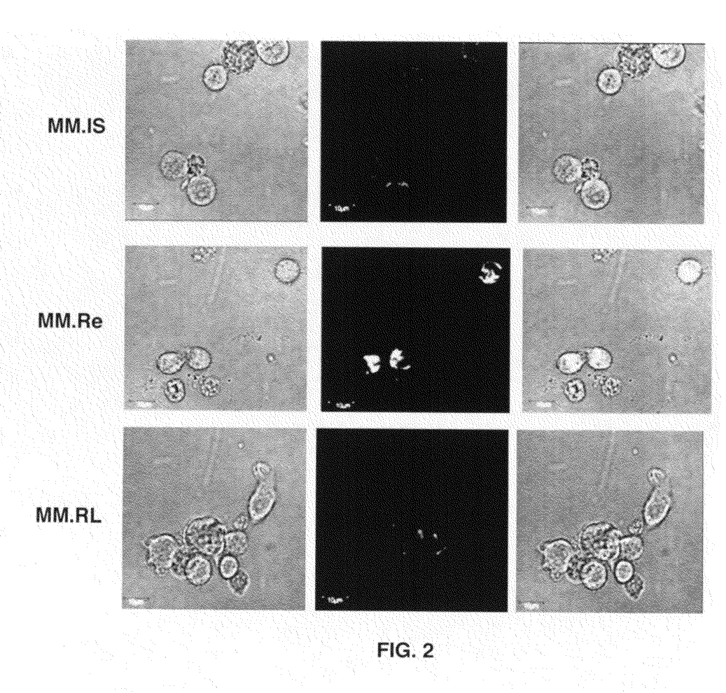

The cellular uptake of AuNP-miR-130b/miR-130b*-Cy3 by multiple myeloma (MM) cells lines, which have different sensitivity to drugs called drug sensitive cells (MM.1S), drug resistant cells (MM.Re), and drug late resistant cells (MM.RL). Three-dimensional confocal microscopy images (shown as a z-stack) of these three different multiple myeloma (MM) cell lines are taken 20 min after exposure to AuNP-miR-130b/miR-130b*-Cy3 are shown in FIG. 2. All three cell lines demonstrate uptake of AuNP-miR-130b/miR-130b*-Cy3

Inhibition of Cancer Cell Proliferation in OVCAR8 Cells and p53-Mutant OVCAR8 Cells

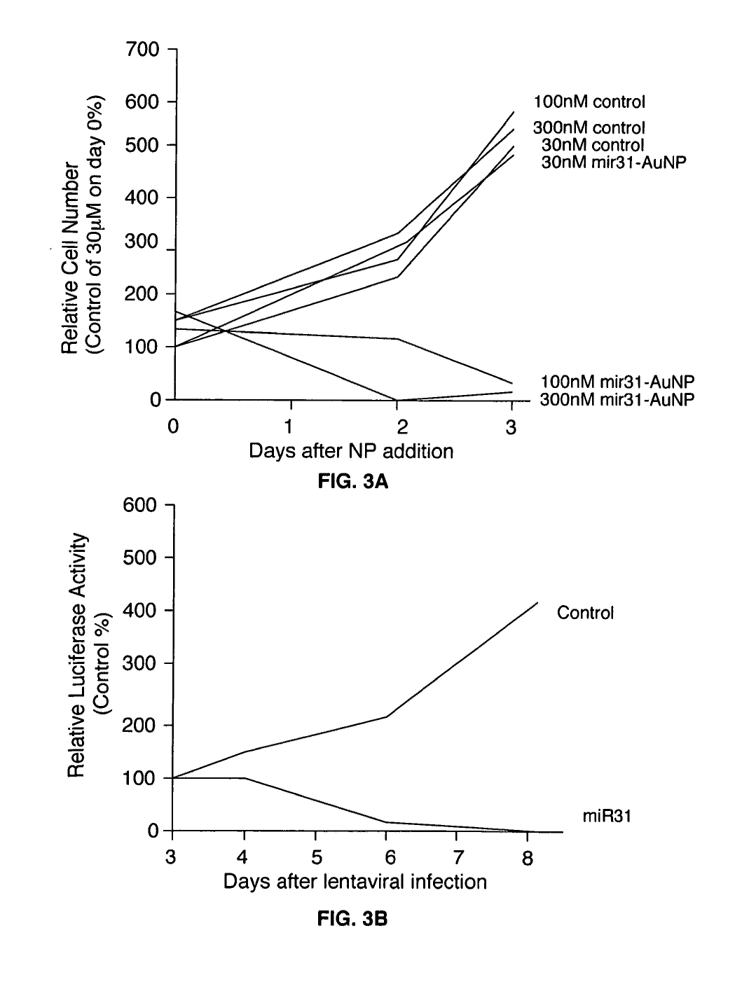

AuNP-miR-31/miR-31*Cy5 inhibits cancer cell proliferation in ovarian OVCAR8 cancer cells. FIG. 3 shows experiments where p53 mutant OVCAR8 ovarian cancer cells are seeded into 96-well plates. After 24 hrs, the culture media is replaced with OptiMEM media containing control or AuNP-miR-31/miR-31* at the concentrations shown. After 24, 48 and 72 hrs, the cell viability is measured using an MTS assay (Promega). Data shows that 300 nM AuNP-miR-31/miR-31*Cy5 achieves greater inhibition compared to the control. Data (mean±s.e.m.; n=3) are normalized to cells treated with OptiMEM only. When compared to lentivirally delivered mir-31 (6-8 days for cell killing), the biologic effects of miR-31-AuNPs occurred much more rapidly (2-3 days for cell killing).

To test the efficacy of miR-31-AuNPs, AuNPs conjugated to a duplex miR-31 mimic in which the antisense strand is conjugated to a fluorescent probe, Cy5. Using these fluorescently labeled mimics, the present invention successfully shows that established ovarian cancer cell lines incubated with AuNP-miR-31/miR31*-Cy5 rapidly internalize these particles without the need for transfection. Application of AuNP-miR-31/miR31*-Cy5 to p53-deficient OVCAR8 cells produces a rapid, dose-dependent decrease in cell viability when compared to identical concentrations of control Au-NPs. Neither AuNP-miR-31/miR31*-Cy5 nor control AuNPs kill p53 wild-type HEY ovarian cancer cells. Note that the concentrations of AuNPs used to accomplish significant cell killing is at nanomolar concentrations or less.

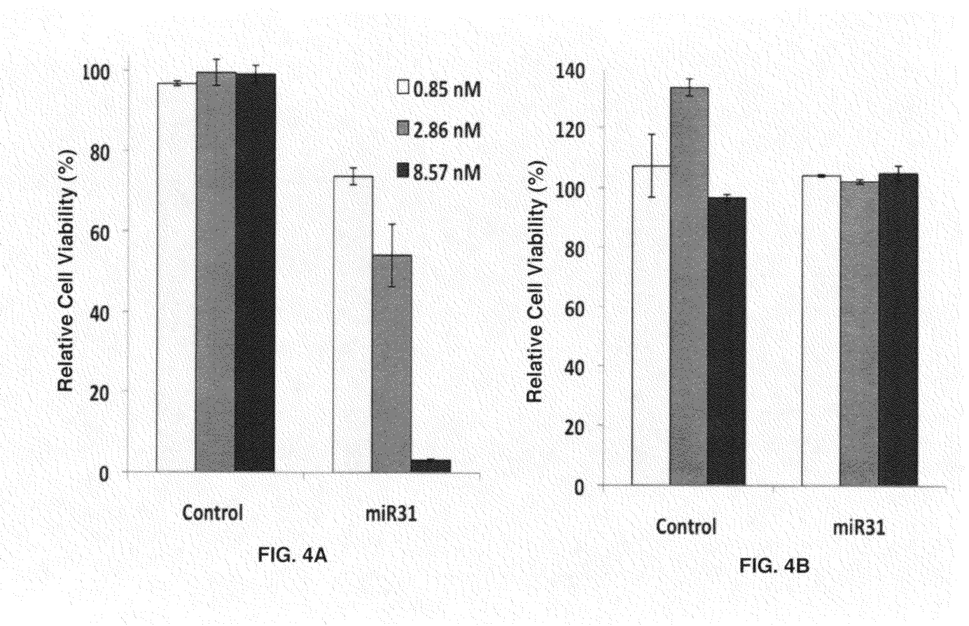

AuNP-miR-31/miR-31*Cy5 selectively kill p53-mutant ovarian cancer cells (OVCAR8 cells). p53 mutant OVCAR8 ovarian cancer cells (FIG. 4A) and p53 wild-type Hey ovarian cancer cells (FIG. 4B) are seeded into 96-well plates. After 24 hrs, the culture media is replaced with OptiMEM media containing control or AuNP-miR-31/miR-31* at the concentrations shown. After 24 hrs, the cell viability is measured using an MTS assay (Promega). Cell viability data (mean±s.e.m.) are normalized to OVCAR8 or HEY ovarian cancer cells treated with OptiMEM media only.

The successful delivery of miR-31 as described herein represents a major therapy for the treatment of women with ovarian cancer. Furthermore, since normal cells in the peritoneum are wild type for p53 and CDKN2A, the data disclosed herein indicate that the intraperitoneal delivery of miR-31 should have minimal toxicity and few side effects. The significantly enhanced efficacy of miR-31/miR31*-Cy5-conjugated gold nanoparticle on cell killing suggests that AuNPs conjugated with validated tumor suppressor miRNAs could be promising therapeutic agents in ovarian and other cancers with significant advantages over other methods currently available for delivering miRNA intracellularly. The results shown in FIGS. 4A-4B show that AuNPs-miR-31/miR31*-Cy5 retain the ability to selectively kill p53-deficient ovarian cancer cells.

### Example 5

### Synthesis of IFHD-Au NP Combinatorial Drug Delivery Systems

### Non-Cytotoxic Cystamine Core PAMAM Dendrimers with Tertiary Amine Interior Groups and tri(hydroxymethyl)amidomethane Surface Groups

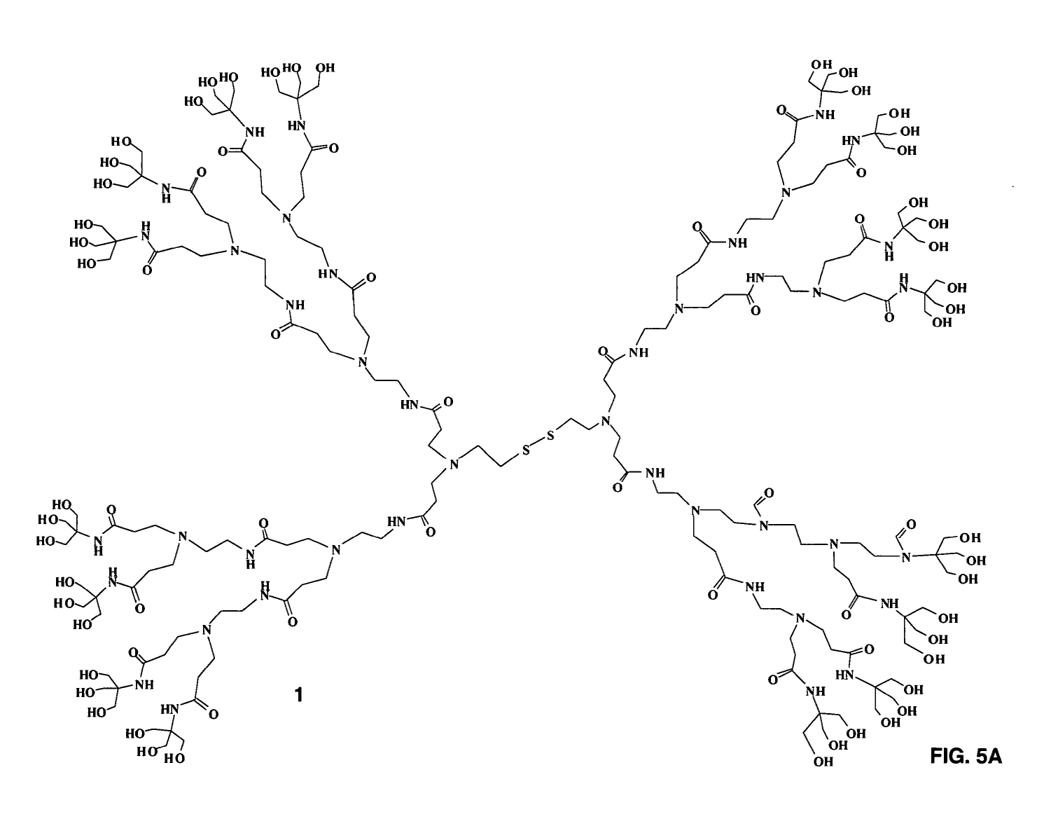

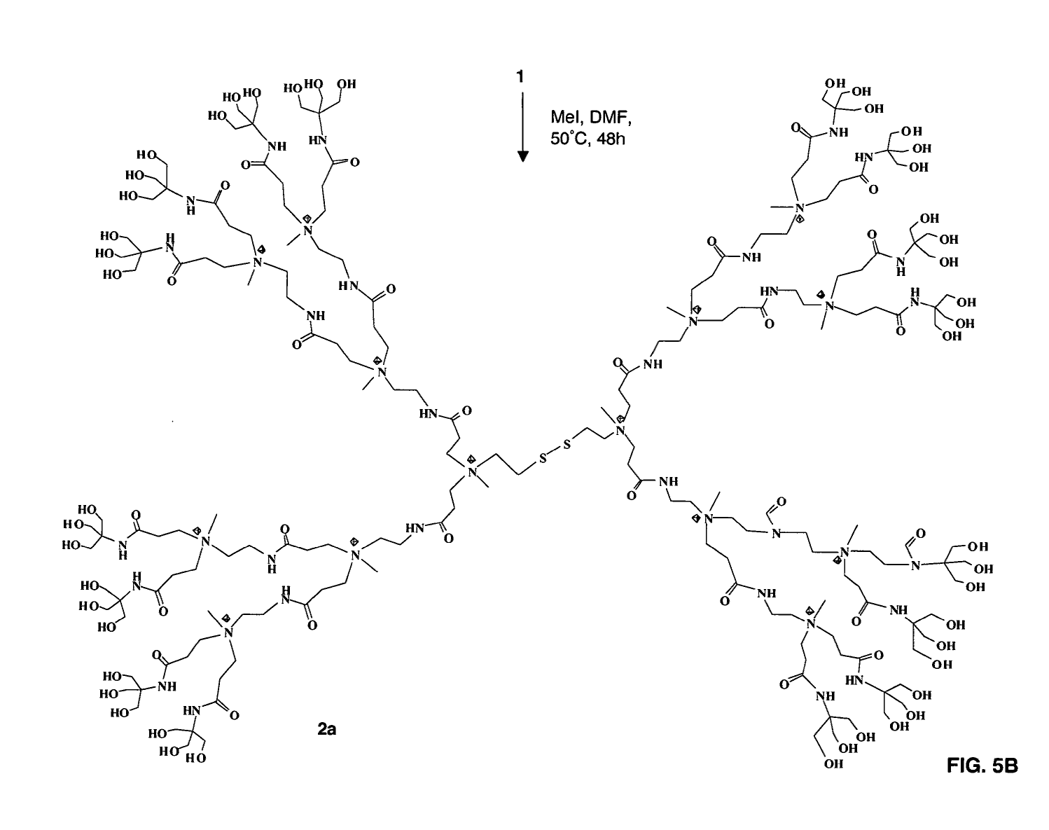

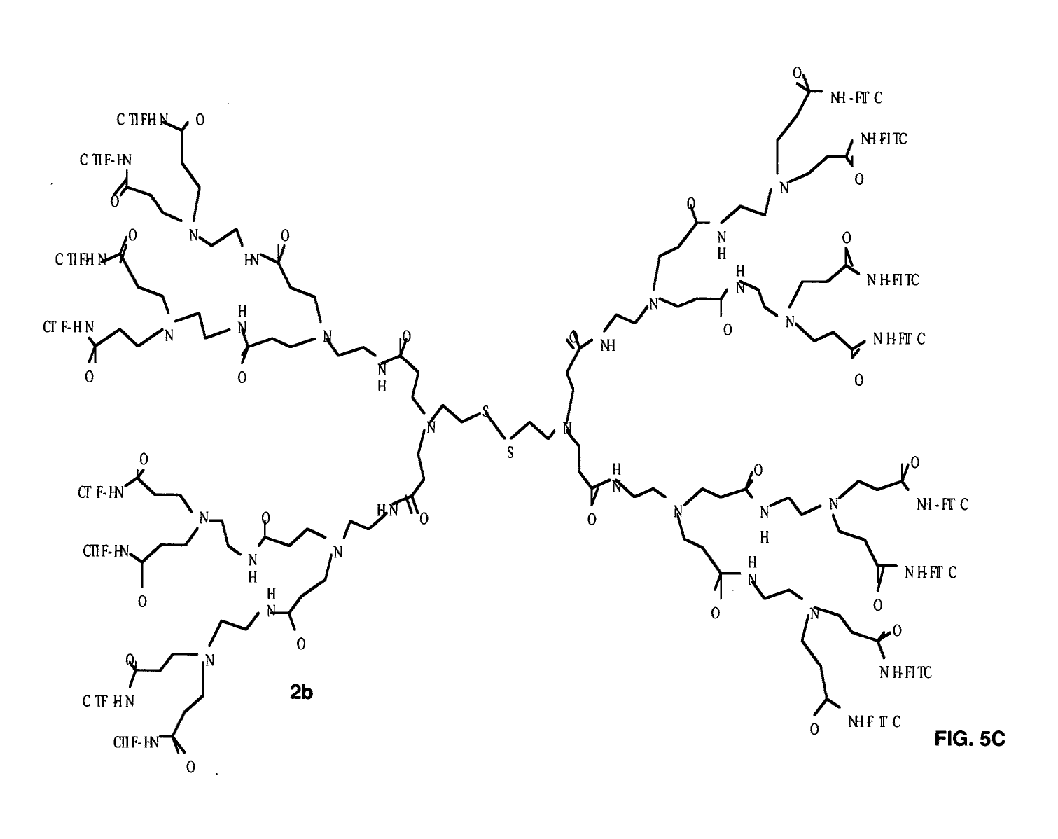

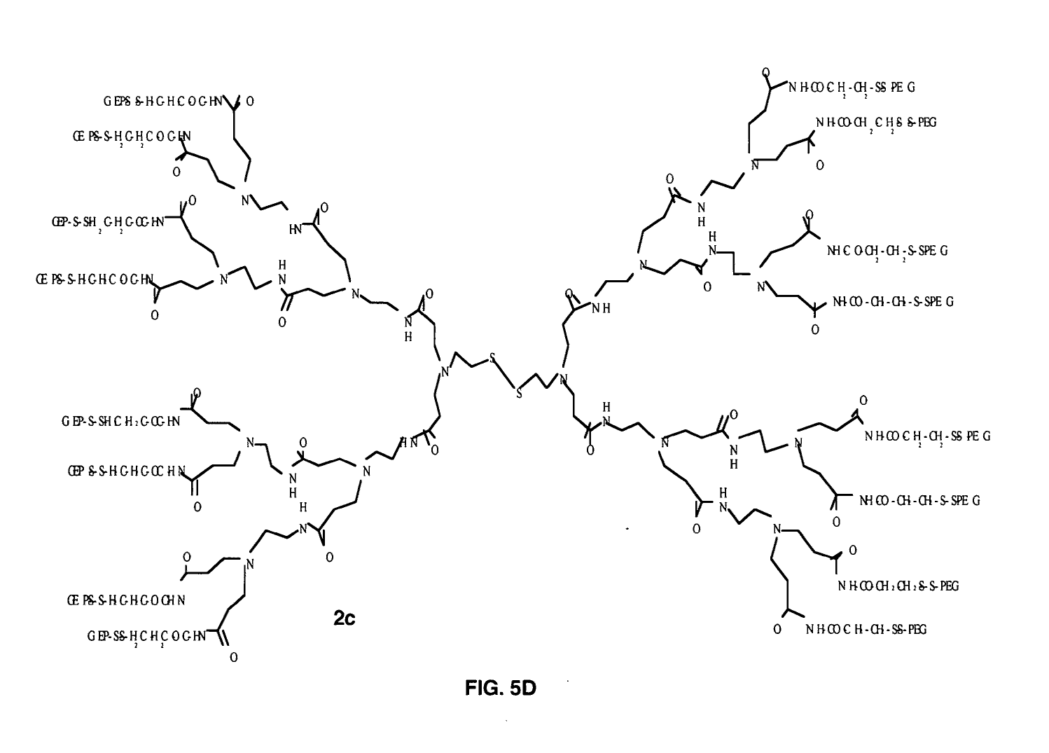

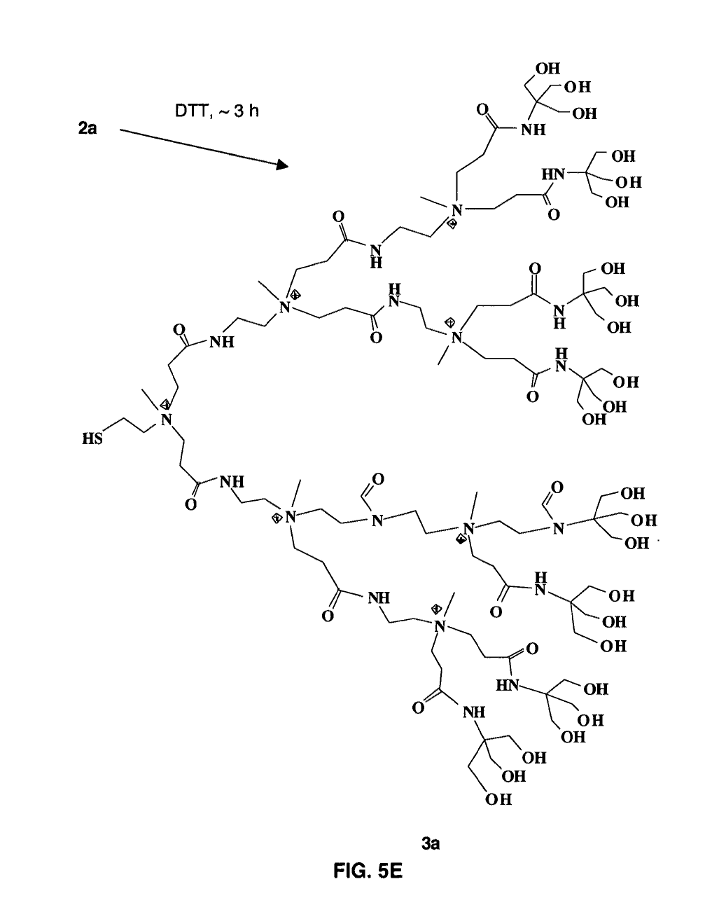

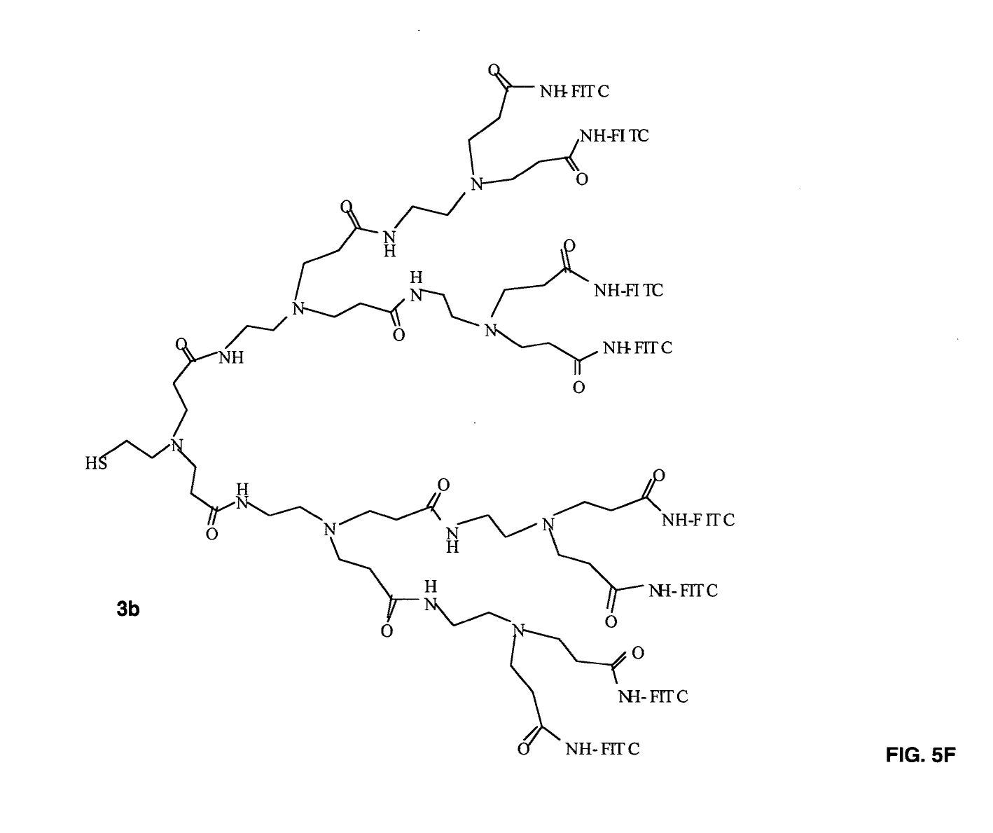

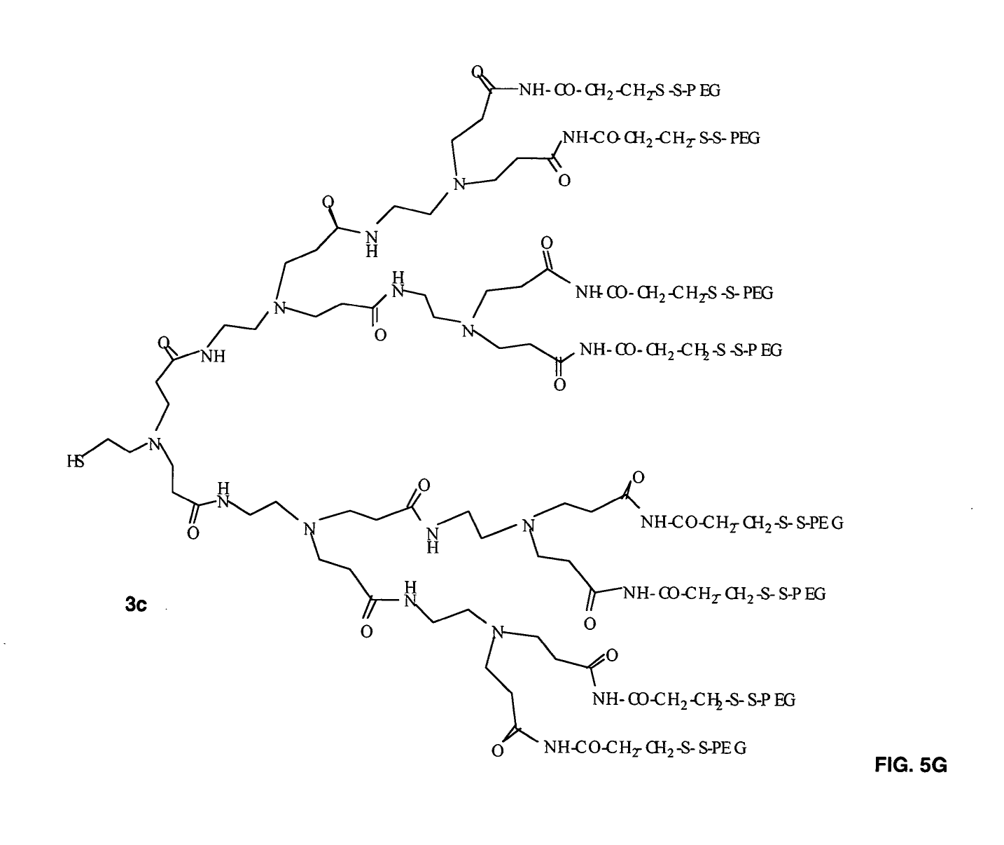

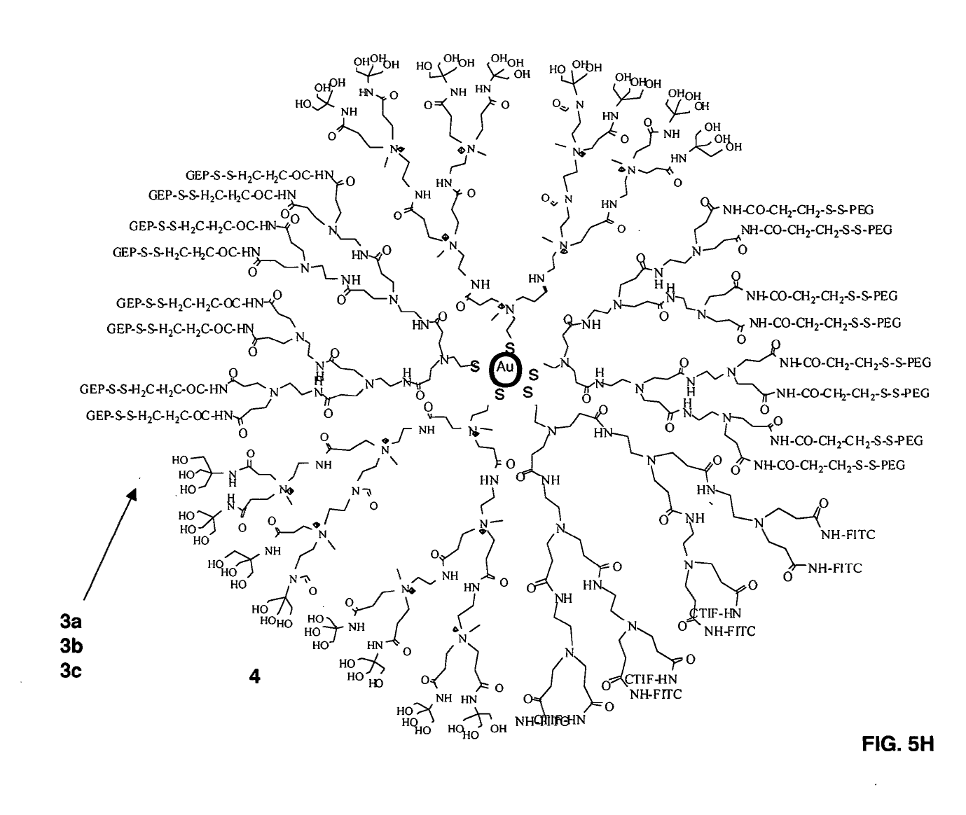

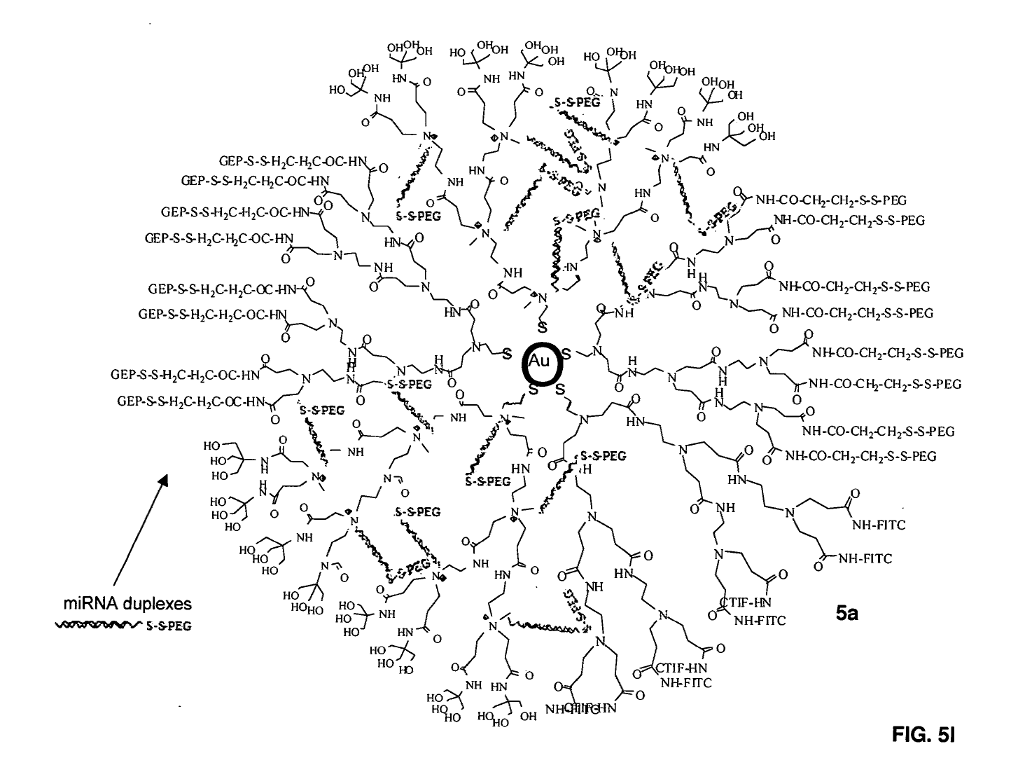

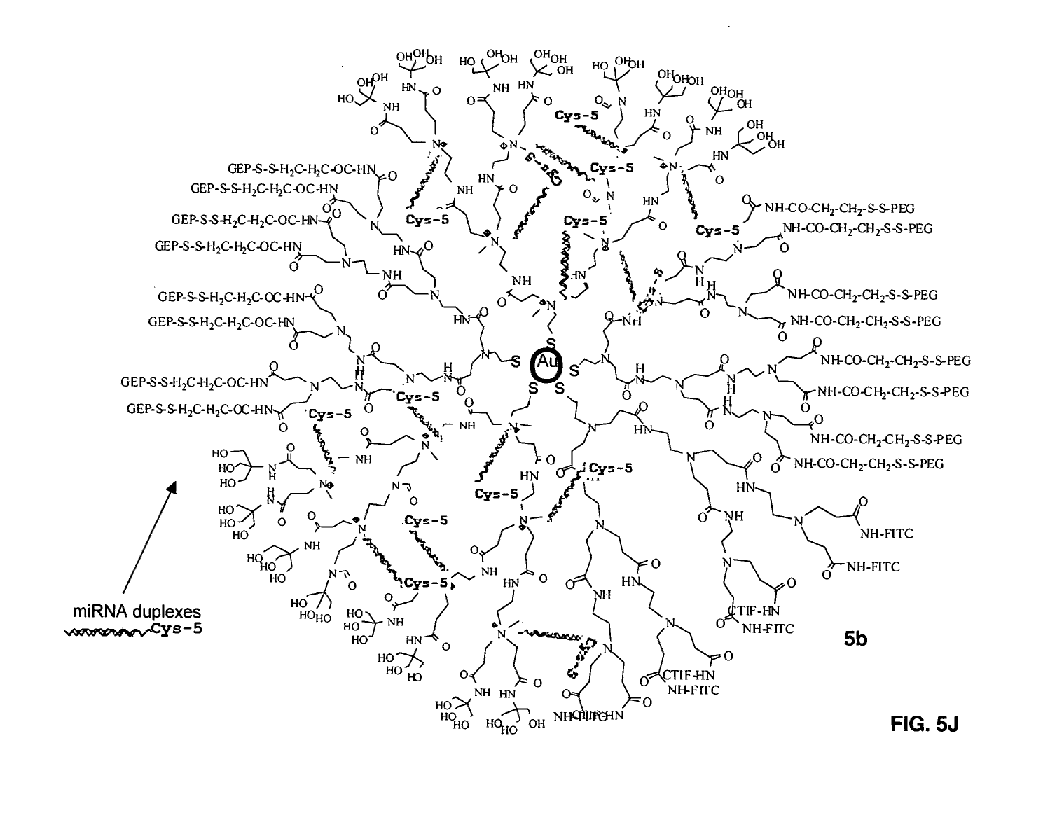

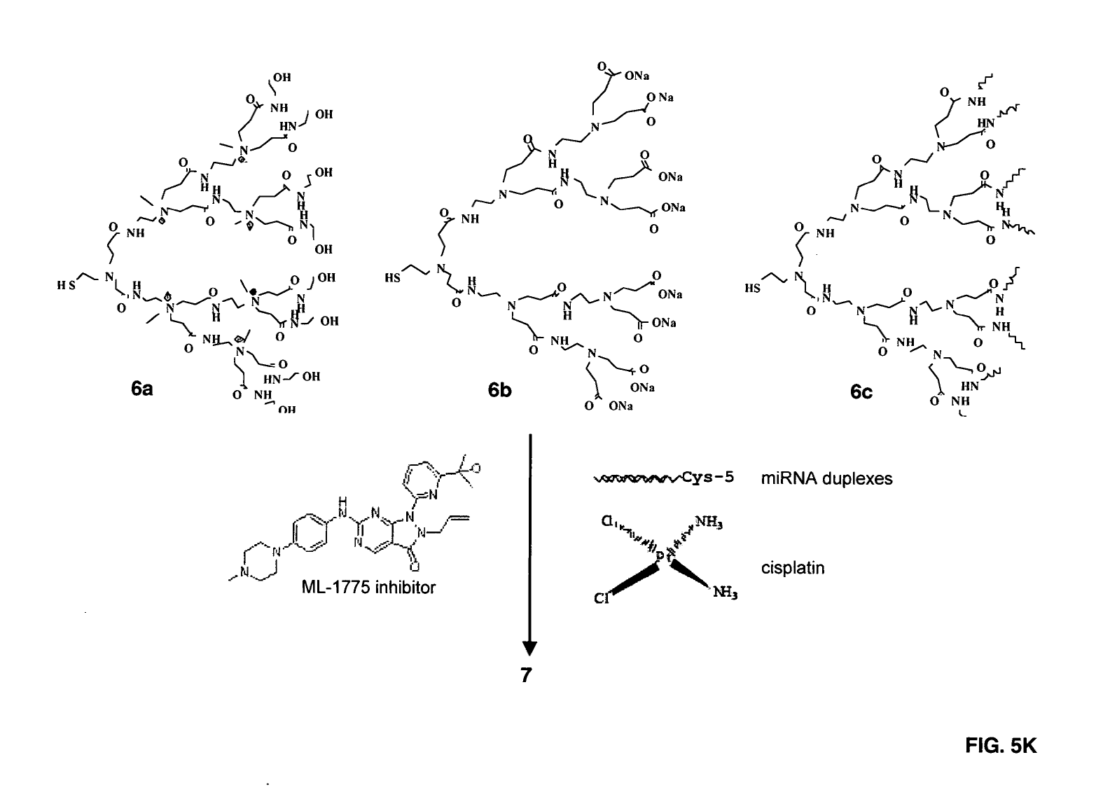

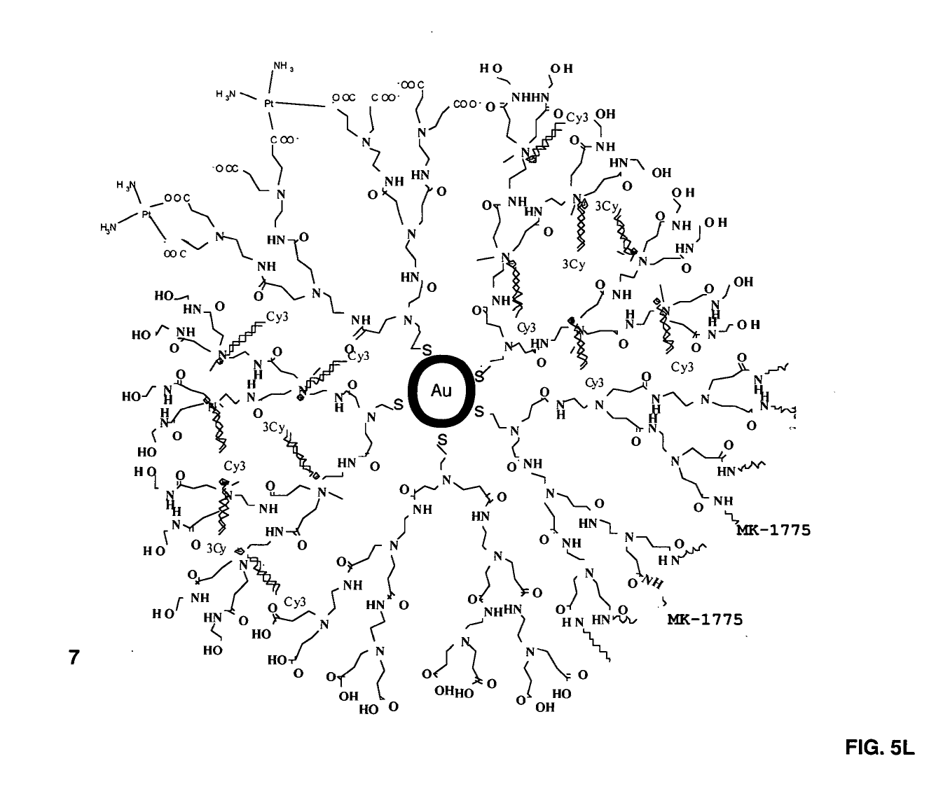

miRNA duplexes such as but not limited to miRNA/miRNA*-S—S-polyethylene glycol (PEG) as fluorescein isothiocyanete (FITC)-conjugated thiolated dendrons are attached to the Au NP surface together with miRNA/miRNA*-S—S-polyethylene glycol (PEG)-conjugated thiolated dendrons to reduce the cytotoxicity that occurs in the use of miRNA duplexes with Cy3 or Cy5 tags. Commercially available non-cytotoxic cystamine core PAMAM (generation 1.5 or G1.5) dendrimers with tertiary amine interior groups and tri(hydroxymethyl)amidomethane surface groups 1 is shown in FIG. 5A. In the present invention, these tertiary amine interior groups in cystamine core PAMAM (G1.5 or G2.5 or G3.5 or G4.5 or G5.5 or higher half generations) dendrimers are internally quaternized to create positive charge to form a stable electrostatic interactions with negatively charged miRNA/miRNA*-S—S-polyethylene glycol) (PEG), as an example, miR-130b/miR-130b*-S—S-PEG or miR-31/miR-31*-S—S-PEG or any other miRNA duplex-S—S-PEG of interest, or miRNA/miRNA*-Cy3 or Cy5, as an example, miR-130b/miR-130b*-Cy3 or miR-31/miR-31*-Cy5 or any other miRNA duplex-Cy3 or Cy5 of interest.

One of the most interesting forms of miRNA duplexes in this invention includes miRNA/miRNA*-S—S-polyethylene glycol (PEG) as fluorescein isothiocyanete (FITC)-conjugated thiolated dendrons are attached to the Au NP surface together with miRNA/miRNA*-S—S-polyethylene glycol (PEG)-conjugated thiolated dendrons to reduce the cytotoxicity that occurs in the use of miRNA duplexes with Cy3 or Cy5 tags. The cystamine core PAMAM (G1.5 or G2.5 or G3.5 or G4.5 or G5.5 or higher half cegenerations) dendrimers with same tertiary amine interior groups but with different surface groups such as, but not limited to, amino, sodium carboxylate, amido ethanol, succinamic acid, hexylamide, amidoethylethanolamine, are used to chemically conjugate anticancer drugs or small molecule inhibitors directly on to the surface groups or by modifying the therapeutic agents with appropriate functionality to accommodate them into the scaffolds.

### Cystamine Core PAMAM (G1.5 or G2.5 or G3.5 or G4.5 or G5.5 or Higher Half Cegenerations) Dendrimers

The methodology used with non-cytotoxic cystamine core PAMAM dendrimers is extended to cystamine core PAMAM (G1.5 or G2.5 or G3.5 or G4.5 or G5.5 or higher half cegenerations) dendrimers with desirable nano-cavities to physically absorb anticancer drugs and small molecule inhibitors. Although thiolated hyperbranced dendrons with tertiary amine interior groups are considered herein due to their biocompatibility, different interior groups with biocompatibility are also desirable in this invention. Internal quaternization of tertiary amine interior groups in non-cytotoxic cystamine core PAMAM (generation 1.5 or G1.5) dendrimers with tri(hydroxymethyl)amidomethane surface groups (3a) (FIG. 5E) facilitates conjugation of negatively charged miRNA/miRNA*-S—S-poly(ethylene glycol) (PEG), e.g., miR-130b/miR-130b*-S—S-PEG or miR-31/miR-31*-S—S-PEG or any other miRNA duplex-S—S-PEG of interest, or miRNA/miRNA*-Cy3 or Cy, e.g., miR-130b/miR-130b*-Cy3 or miR-31/miR-31*-Cy5 or any other miRNA duplex-Cy3 or Cy5 of interest, via stable electrostatic interactions. The same procedure is followed to internally quaternize tertiary amine interior groups in cystamine core PAMAM dendrimers with G1.5 or G2.5 or G3.5 or G4.5 or G5.5 or higher half cogenerations. The same procedure is followed to internally quaternize tertiary amine interior groups in cystamine core PAMAM dendrimers with different surface groups, such as amino, sodium carboxylate, amido ethanol, succinamic acid, hexylamide, amidoethylethanolamine etc. and also with different nano-cavitives. The cystamine core PAMAM (G1.5 or G2.5 or G3.5 or G4.5 or G5.5 or higher half generations) dendrimers with same tertiary amine interior groups and amine surface groups are used to conjugate FITC (2b) (FIG. 5C). The cystamine core PAMAM (G1.5 or G2.5 or G3.5 or G4.5 or G5.5 or higher half generations) dendrimers with same tertiary amine interior groups and amine surface groups are used to conjugate poly(ethylene glycol) (3c) (FIG. 5G) (mPEG-SH (Mw=5000)) via a disulfide linkage to the surface amine groups in the presence of N-succinimidyl-3-(2-pyridyldithio) propionate (SPDP). The cystamine core PAMAM (G1.5 or G2.5 or G3.5 or G4.5 or G5.5 or higher half generations) dendrimers with same tertiary amine interior groups, but different surface groups are used to conjugate anticancer drugs or small molecule inhibitors.

FITC-conjugated cystamine core PAMAM (G1.5) dendrimer with the same tertiary amine interior groups (2b) and PEG-conjugated cystamine core PAMAM (G1.5) dendrimer with same tertiary amine interior groups (2c) are shown for the clarity. The cystamine core PAMAM (G1.5 or G2.5 or G3.5 or G4.5 or G5.5 or higher half generations) dendrimers with same tertiary amine interior groups, but different surface groups are used to conjugate anticancer drugs or small molecule inhibitors.

Cleavage of Non-Cytotoxic Internally Quaternized Cystamine Core PAMAM (Generation 1.5 or G1.5) Dendrimers with tri(hydroxymethyl)amidomethane Surface Groups

In a similar procedure, non-cytotoxic internally quaternized cystamine core PAMAM (generation 1.5 or G1.5) dendrimers with tri(hydroxymethyl)amidomethane surface groups are cleaved into two thiolated internally quaternized dendrons (3a) (FIGS. 5E-5G). The same procedure is followed to cleave cystamine core PAMAM dendrimers with different surface groups such as amino, sodium carboxylate, amido ethanol, succinamic acid, hexylamide, amidoethylethanolamine, etc. and also with different nano-cavitives. FITC-conjugated thiolated dendrons (3b) (FIG. 5F) and PEG-conjugated thiolated dendrons (3c) (FIG. 5G) are shown for clarity. In the same way, anticancer drugs or small molecule inhibitor-conjugated cystamine core PAMAM (G1.5 or G2.5 or G3.5 or G4.5 or G5.5 or higher half generations) dendrimers are cleaved into two thiolated anticaner drug-conjugated dendrons or small molecule inhibitor-conjugated thiolated dendrons.

Conjugation of Internally Quaternized Thiolated Dendrons (G1.5) with tri(hydroxymethyl)amidomethane Surface Groups, FITC-Conjugated Thiolated Dendrons, and PEG-Conjugated Thiolated Dendrons to a Gold Nanoparticle Surface

The conjugation of internally quaternized thiolated dendrons (G1.5) with tri(hydroxymethyl)amidomethane surface groups, FITC-conjugated thiolated dendrons, and PEG-conjugated thiolated dendrons on to a Au NP surface to obtain IFHD-AuNPs is shown in FIG. 4D. First, internally quaternized thiolated dendrons (or FITC- or PEG-conjugated thiolated dendrons or anticancer drug- or small molecule inhibitor-conjugated thiolated dendrons) and Au NPs are mixed in various weight ratios (0, 1.25, 2.5, 5, 10, 20, and 30) or concentration ratios and incubated for 1 hr. The number of internally quaternized thiolated dendrons conjugated to a Au NP surface is calculated by destroying Au NPs using KCN and monitoring the ¹H-NMR spectroscopy. Here, the number of dendrons in the solution is monitored by calculating the peak concentration relevant to interior groups or surface groups and then the number of thiolated dendrons per nanoparticle is calculated by dividing the concentration of dendrons by the concentration of Au NPs. Considering the possible chemical reactivity of certain functional groups of dendrons with KCN, other appropriate chemicals such as dithiothreitol (DTT) or mercapto-alkanes with different functional end groups are used to displace the thiolated dendrons from the Au NP surface. Knowing the maximum payload density of thiolated dendrons on a Au NP, determined from the process of preparing IFHD-Au NPs, thiolated internally quaternized dendrons, FITC-conjugated thiolated dendrons, and PEG-conjugated thiolated dendrons are conjugated to the Au NP surface with different concentration or weight ratios.

In the process of preparing IFHD-Au NPs, anticancer drug-conjugated thiolated dendrons or small molecule inhibitor-conjugated thiolated dendrons, FITC-conjugated thiolated dendrons and PEG-conjugated thiolated dendrons are conjugated to the Au NP surface with different concentration or weight ratios. In the process of preparing IFHD-Au NPs, internally quaternized thiolated dendrons with tri(hydroxymethyl)amidomethane surface groups, FITC-conjugated thiolated dendrons, PEG-conjugated thiolated dendrons, anticancer drug-conjugated thiolated dendrons and small molecule inhibitor-conjugated thiolated dendrons are conjugated onto nanoparticle surface together to create a delivery system that can carry mirRNA duplexes, anticancer drugs and small molecule inhibitors simultaneously. The concentration ratio between nanoparticles and thiolated dendrons with therapeutic agents of interest can be varied in order to conjugate low, moderate or higher number of therapeutic agent of interest. The same procedures are used to prepare IFHD-AuNPs with thiolated dendrons with G1.5 or G2.5 or G3.5 or G4.5 or G5.5 or higher half generations.

Conjugation of miRNA/mRA*-S—S-PEG or -Cy3 or Cy5 Duplexes to IFHD-AuNPs

Thiolated dendrons with G1.5 or G2.5 or G3.5 or G4.5 or G5.5 or higher half generations are utilized to prepare IFHD-AuNPs as in FIG. 5H. Conjugation of miRNA/miRNA*-S—S-polyethylene glycol) (PEG) duplexes, e.g, miR-130b/miR-130b*-S—S-PEG or miR-31/miR-31*-S—S-PEG or any other miRNA duplex-S—S-PEG of interest or miRNA/miRNA*-Cy3 or Cy5 duplexe, e.g., miR-130b/miR-130b*-Cy3 or miR-31/miR-31*-Cy5 or any other miRNA duplex-Cy3 or Cy5 of interest, to IFHD-AuNP is shown in FIGS. 5I-5J. In the synthesis process of miRNA-conjugated IFHD-AuNPs, miRNAs are conjugated in two different ways. In one method cystamine core PAMAM (G1.5 or G2.5 or G3.5 or G4.5 or G5.5 or higher half generation) dendrimers with tri(hydroxymethyl)amidomethane surface groups are internally quaternized (FIGS. 5B-5D), cleaved into two thiolated-dendrons (FIGS. 5E-5G) and immediately conjugated to the Au NP surface (FIG. 5H) and then finally miRNA/miRNA*-S—S-PEG duplexes or miRNA/miRNA*-Cy3 or Cy5 duplexes are conjugated to the IFHD-AuNPs or cystamine core PAMAM (G1.5 or G2.5 or G3.5 or G4.5 or G5.5 or higher half generation) dendrimers with tri(hydroxymethyl)amidomethane surface groups are internally quaternized (FIGS. 5B-5D), cleaved into two thiolated-dendrons (FIG. 5E-5G) and immediately incubated in different weight ratios of Au NPs and miRNA/miRNA*-S—S-PEG duplexes or miRNA/miRNA*-Cy3 or Cy5 duplexes in order to construct miRNA duplexes-conjugated IFND-Au NPs.

Conjugation of miRNA/miRNA*-S—S-poly(ethylene glycol) (PEG) Duplexes

FIGS. 5I-5J depict the conjugation of miRNA/miRNA*-S—S-polyethylene glycol) (PEG) duplexes (5a) (as an example, miR-130b/miR-130b*-S—S-PEG or miR-31/miR-31*-S—S-PEG or any other miRNA duplex-S—S-PEG of interest) or miRNA/miRNA*-Cy3 or Cy5 duplexes (5b) (as an example, miR-130b/miR-130b*-Cy3 or miR-31/miR-31*-Cy5 or any other miRNA duplex-Cy3 or Cy5 of interest) to as prepared IFHD-AuNP 4 in FIG. 5H. In the synthesis process of miRNA-conjugated IFHD-AuNPs, miRNAs are conjugated in two different ways. In one method, cystamine core PAMAM (G1.5 or G2.5 or G3.5 or G4.5 or G5.5 or higher half generation) dendrimers with tri(hydroxymethyl)amidomethane surface groups are internally quaternized (FIGS. 5B-5D), cleaved into two thiolated-dendrons (FIGS. 5E-5G) and immediately conjugated to the Au NP surface (FIG. 5D) and then finally miRNA/miRNA*-S—S-PEG duplexes or miRNA/miRNA*-Cy3 or Cy5 duplexes are conjugated to the IFHD-AuNPs. In another method cystamine core PAMAM (G1.5 or G2.5 or G3.5 or G4.5 or G5.5 or higher half generation) dendrimers with tri(hydroxymethyl)amidomethane surface groups are internally quaternized (FIG. 5B), cleaved into two thiolated-dendrons (FIG. 5C) and immediately incubated in different weight ratios of Au NPs and miRNA/miRNA*-S—S-PEG duplexes or miRNA/miRNA*-Cy3 or Cy5 duplexes in order to construct miRNA duplexes-conjugated IFND-Au NPs.

In the synthetic process for anticancer drug-conjugated IFHD-AuNPs or small molecule inhibitor-conjugated IFHD-Au NPs, as an example, cisplatin anticancer drug also is conjugated in two different ways. In one method, cystamine core PAMAM (G1.5 or G2.5 or G3.5 or G4.5 or G5.5 or higher half generation) dendrimers with amine (or sodium carboxylate) surface groups are cleaved into two thiolated-dendrons and immediately conjugated to the Au NP surface and then finally cisplatin is conjugated to the IFHD-AuNPs. In another method cystamine core PAMAM (G1.5 or G2.5 or G3.5 or G4.5 or G5.5 or higher half generation) dendrimers with amine (or sodium carboxylate) surface groups are cleaved into two thiolated-dendrons and immediately incubated in different weight ratios of Au NPs and cisplatin in order to construct cisplatin-conjugated IFND-Au NPs. The same two different methods are followed to conjugate small molecule inhibitors like MK-1775.

The payload density of therapeutic agents in the two different methods are encountered to load higher, medium or lower density of therapeutic agent in a IFHD-Au NP system. In order to conjugate one or more therapeutic agents to IFHD-Au NPs, cystamine core PAMAM (G1.5 or G2.5 or G3.5 or G4.5 or G5.5 or higher half generation) dendrimers with tri(hydroxymethyl)amidomethane surface groups are internally quaternized, cleaved into two thiolated-dendrons (A=maximum loading of thiolated dendrons with tri(hydroxymethyl)amidomethane surface groups on a Au NP surface) and at the same time cystamine core PAMAM (G1.5 or G2.5 or G3.5 or G4.5 or G5.5 or higher half generation) dendrimers with amino (or sodium carboxylate) surface groups are cleaved into two thiolated-dendrons (B=maximum loading of thiolated dendrons with amino (or sodium carboxylate) surface groups on a Au NP surface) and immediately incubated with Au NPs in the ratio of 1:xA:yB (x=0 to 0.5 and y=0 to 0.5). This ratio is varied depending on the specific target of the interest.

The dispersion stability of therapeutic agent-conjugated IFHD-AuNPs in vitro and in vivo application is enhanced by covering uncovered surface areas of Au NPs by 1) conjugating thiolated oligoethylene glycol (OEG); 2) by conjugating thiolated dendrons with OEG or polyethylene glycol (PEG) spacer units to the surface of Au NPs (in this case payload density of therapeutic agent-conjugated dendrons is lowered in order to give the space for the thiolated dendrons with OEG or PEG spacer units); 3) PEG-conjugated thiolated dendrons (in this case too much of a payload density of therapeutic agent-conjugated dendrons is lowered in order to give the space for the thiolated dendrons with hexylamine surface groups); or 4) conjugating miRNA/miRNA*-S—S-PEG duplexes to internally quartenized amine groups in thiolated dendrons.

Simultaneous Conjugation of PEG- or CY3 or Cy5-Modified miRNA Duplexes, Cisplatin and MK-1775 to IFHD-AuNP

miRNA/miRNA*-S—S-PEG or miRNA/miRNA*-Cy3 or Cy5 duplexes, cisplatin and small molecule inhibitors are conjugated to the IFHD-AuNPs 6a,6b,6c in the ratio of xC:yD:zE where x, y, z will vary from 0 to 0.5 or higher (x=0 to 0.5 and y=0 to 0.5) depending on the specific target of interest to form 7 (FIGS. 5K-5L). FITC-conjugated thiolated dendrons and PEG-conjugated thiolated dendrons must be conjugated to the same IFHD-Au NP system as described herein.

Synthesis of Gold Nanoparticles and miRNA-Conjugated Gold Nanoparticles

Citrate-stabilized gold (Au) nanoparticles (NPs) with sizes from 13, 30, 50, 150, and 250 nm in diameter, are prepared using the Frens method (1). Following the synthesis, RNase free Au NPs are prepared by treating with 0.1% diethylpyrocarbonate (DEPC) for 12 h with stirring, then autoclaved at 121° C. for 1 h (2). miRNA-conjugated Au NPs are synthesized following the method reported by the D. A. Giljohann et al. (2), briefly, Au NPs are treated with 0.1% diethylpyrocarbonate (DEPC) overnight with stirring, then autoclaved at 121° C. for 1 h. Pre-formed, thiolated RNA duplexes (1000 nM) are incubated with the Au NPs (10 nM) which has been adjusted to 0.1 M NaCl. The mixture is aged in solutions with increasing concentration from 0.1 M to 0.3 M and sonicated. Oligoethylene glycol (30 μmol/mL) is added 24 h after duplex addition. The miRNA-AuNPs are purified by centrifugation at (13,000 rpm, 20 min.) at 4° C., and resuspended in sterile phosphate buffer saline (PBS137 mM NaCl, 10 mM Phosphate, 2.7 mM KCl, Ph 7.4). That process is repeated three times (2).

### Synthesis of Internally Quaternized Dendrons, or Sn.QPAMAM-R

Cystamine core PAMAM dendrimers with different surface groups as well as various generations are internally quaternized by following the method that described to internally quaternized PAMAM-OH (3-4). As an example, generation 2.5 cystamine core PAMAM dendrimers with tris(hydroxylmethyl) amidomethane-terminated surface groups (S—S2.5.PAMAM-NHC(OH)₃) (0.134 mg, 0.015 mmol) is dissolved in N,N′-dimethylformamide (DMF, 1 mL) and equivalent moles of methyl iodide relative to interior tertiary amine in S—S2.5.PAMAM-NHC(OH)₃, diluted in DMF (0.5 mL) is added into the mixture. The reaction mixture is sealed and stirred at 50° C. for 48 h. After 48 h, the reaction mixture is precipitated into diethylether and vacuum dried. The resulting solid is redissolved in water (1 mL) and then purified by dialysis against 2M NaCl and deionized water successively using Spectra/Por dialysis membrane with an MWCO 6000-8000. It is then lyophilized to obtain the pure solid. Then, the internally quatenized dendrimer, S—S2.5.QPAMAM-NHC(OH)₃ are reduced using DTT for ˜3 h in PBS and filtered through NAP-5 (Sephadex G-25 DNA grade) columns.

### Synthesis and Characterization of Sn.QPAMAM-R-Conjugated Au NPs or IFHD-AuNPs

RNase free citrate-stabilized Au NPs are mixed with SnQPAMAM-R dendrons at various weight ratios of gold nanoparticles to SnQPAMAM-R (5, 10, 20, and 30, 40 and 50). After 15 min incubation, electrophoretic mobility of the mixtures is visualized on a 2% (w/v) agarose gel. It is carried out for 50 min at 100V in TAE buffer solution (40 mM Tris-HCl, 1% (v/v) acetic acid, 1 mM EDTA), and the bands are stained with ethidium bromide.

Synthesis and Characterization of miRNA/miRNA*Cy3 or Cy5 and OEG-Conjugated IFHD-AuNPs

The SnQPAMAM-R-conjugated Au NPs are modified with miRNA/miRNA*Cy3 duplexes and oligoethylene glycol (OEG). Briefly, 1 μg of Sn.QPAMAM-R-conjugated Au NPs are incubated with miRNA/miRNA*Cy3 duplexes at various weight ratios of Sn.QPAMAM-R-conjugated Au NPs to miRNA/miRNA*Cy3 duplexes (30, 60, 90, 120, 150, 180, and 210). After 15 min incubation, OEG (30 μmol/mL) will be added and incubated for 30 min. Then, the electrophoretic mobility of the mixtures is visualized on a 2% (w/v) agarose gel. It is carried out for 50 min at 100V in TAE buffer solution (40 mM Tris-HCl, 1% (v/v) acetic acid, 1 mM EDTA), and the bands are stained with ethidium bromide.

Synthesis and Characterization of miRNA/miRNA*3′-PEG-Conjugated IFHD AuNPs. miRNA/miRNA*-PEG Duplexes

The duplexes are prepared following a published procedure (5). Following the synthesis, Sand nQPAMAM-R-conjugated Au NPs and miRNA/miRNA*-PEG duplexes are incubated at various weight ratios of Sn.QPAMAM-R-conjugated (30, 60, 90, 120, 150, 180, and 210). After 15 min incubation, electrophoretic mobility of the mixtures are visualized on a 2% (w/v) agarose gel. It is carried out for 50 min at 100V in TAE buffer solution (40 mM Tris-HCl, 1% (v/v) acetic acid, 1 mM EDTA), and the bands are stained with ethidium bromide.

### Synthesis and Characterization of Fluorescein Isothiocyanete(FITC)-Conjugated IFHD-AuNPs

FITC-NH(PAMAM)-Sn is prepared and attached to miRNA/miRNA*3′-PEG-conjugated NIFD Au NPs or miRNA/miRNA*Cy3 or Cy5-conjugated NIFD Au NPs or drugs-conjugated Au NPs in order to explore the intercellular uptake and leaving the cells after delivering miRNAs or drugs or both. FITC and S—SnPAMAM-NH₂ are dissolved in PBS, pH 7.4. FITC solution is added slowly to the stirring S—SnPAMAM-NH₂ solution (S—SnPAMAM-NH₂:FITC molar ratio 1:1.2) at room temperature and incubated for 24 h in the dark with stirring. The resulting mixture is purified by dialysis against deionized water successively using Spectra/Por dialysis membrane with an MWCO 1350 or 3500 until free FITC not detected by thin layer chromatography (TLC, mobile phase chloroform, methanol, and ammonia (5:4:1)). It is then lyophilized to obtain the pure solid. The FITC-NH(PAMAM)-S—Sn are reduced using DTT for ˜3 h in PBS and filtered through NAP-5 (Sephadex G-25 DNA grade) columns. FITC-modified dendrons, FITC-NH(PAMAM)-Sn are conjugated to miRNA/miRNA*3′-PEG-conjugated NIFD Au NPs or miRNA/miRNA*Cy3 or Cy5-conjugated NIFD Au NPs or drugs-conjugated Au NPs by incubating in different weight ratios (3, 6, 9, and 12) for 30 min. at room temperature.

Synthesis and Characterization of Cisplatin-Conjugated IFHD-AuNPs. c,c,t-[Pt(NH₃)₂Cl₂(OH)₂]

Cisplatin-conjugated IFHD-AuNPs. c,c,t-[Pt(NH₃)₂Cl₂(OH)₂] is synthesized according to the literature (6). c,c,t-[Pt(NH₃)₂Cl₂(OH)(O₂CCH₂ CH₂CO₂H] is synthesized following to method published by S. Dhar et al., (8). Then, 0.025 μmol of N-hydroxysuccinimide (NHS) is added to 0.025 μmol 1-ethyl-3-[3-dimethylaminopropyl]carbodiimide (EDC) and added 50 μL of water into the mixture. The number of primary amine groups in Au[Sn.QPAMAM-NH₂]ₓ, that is x multiplies by the number of amines in each generational dendrons (y) are calculated and ½ y number of moles of c,c,t-[Pt(NH₃)₂Cl₂(OH)(O₂CCH₂ CH₂CO₂H] is added to NHS/EDC mixture. Then, y number of moles of Au[Sn.QPAMAM-NH₂], is added to the above reaction mixture and it is stirred for 24 h at room temperature. The un-reacted c,c,t-[Pt(NH₃)₂Cl₂(OH)(O₂CCH₂ CH₂CO₂H] is removed 100 kDa molecular weight cutoff ultracentrifugation filtration membrane (7).

Synthesis and Characterization of Carboplatin-conjugated IFHD-AuNPs. cis-diamine(3-hydroxy-1,1-cyclobutanedicarboxylate-O,O′)platinum(II)

Carboplatin-conjugated IFHD-AuNPs. cis-diamine(3-hydroxy-1,1-cyclobutanedicarboxylate-O,O′)platinum(II) is synthesized according to the literature (8). Then, 0.025 μmmol of N-hydroxysuccinimide (NHS) is added to 0.025 μmol 1-ethyl-3-[3-dimethylaminopropyl]carbodiimide (EDC) and added 50 μL of water into the mixture. The number of primary amine groups in Au[Sn.QPAMAM-NH₂]ₓ, that is x multiplies by the number of amines in each generational dendrons (y) are calculated and ½ y number of moles of cis-diamine(3-hydroxy-1,1-cyclobutanedicarboxylate-O,O′)platinum(II) is added to NHS/EDC mixture. Then, y number of moles of Au[Sn.QPAMAM-NH₂]ₓ is added to the above reaction mixture and it is stirred for 24 h at room temperature. The un-reacted cis-diamine(3-hydroxy-1,1-cyclobutanedicarboxylate-O,O′)platinum(II) is removed 100 kDa molecular weight cutoff ultracentrifugation filtration membrane.

### Synthesis and Characterization of Small Molecule Inhibitors (MK-1775)-Conjugated IFHD-AuNPs

Conjugation of MK-1775 to IFHD-AuNPs can be carried out several different ways; (1) cystamine core PAMAM (G1.5 or G2.5 or G3.5 or G4.5 or G5.5 or higher half generation) dendrimers with carboxylic acid surface groups are cleaved to thiolated dendrons (G1.5 or G2.5 or G3.5 or G4.5 or G5.5 or higher half generation) with carboxylic acid surface groups and then immediately incubated with Au NPs in different weight ratios. The number of thiolated dendrons per Au NPs is calculated by destroying the Au NPs using KCN and then monitoring the ¹H-NMR to find the number of dendrons in the solution and dividing the concentration of dendrons by concentration of NPs. It is noteworthy to mention that considering the possible chemical reactivity of certain functional groups of dendrons with KCN, other appropriate chemicals such as dithiothreitol (DTT) or mercapto-alkanes with different functional end groups are used to displace the thiolated dendrons from the Au NP surface. Then MK-1775 is added to the IFHD-Au NPs with thiolated dendrons with carboxylic acid surface groups with different weight ratios of iFHD-AuNPs: MK-1775 in order to chemically conjugate MK-1775 via ester linkage, MK-1775 is physically adsorbed to the nano-cavities of IFHD-AuNPs according to the published literature (9).

Intracellular Uptake and Target Downregulation of AuNP-miR-130b/miR-130b*-Cy3

To assay the intracellular uptake of AuNP-miRNA/miRNA*Cy3, the microRNA 130b is conjugated to the nanoparticles following the protocol described above to generate miR130b/miR130b*Cy3 AuNPs. Multiple Myeloma cell lines with different sensitivity to drugs; MM.1S sensitive; MM.Re resistant; MM.RL late resistant are seeded on 24 well plates at a density of 1.5×10⁶ cells per well and treated with 30 nM miR130b/miR130b*Cy3 AuNPs. Treatments are performed as follows: miR130b/miR130b*Cy3 AuNPs are resuspended in OptiMeM media (GIBCO) supplemented with 5% of Fetal Bovine Serum (FBS) and adequate volume is added directly to the cells on the well to reach a final concentration of 30 nM, cells are incubated at 37° C. in 5% CO₂; After 20 min. a small aliquot is taken and used to performed live-cell imaging using confocal microscopy. Cells are loaded in a neubauer hemocytometer chamber and the number of total cells and Cy3 positive cells are counted, 88.93% of the cells showed positive signal (FIG. 2). The rest of the cells are incubated at 37° C. in 5% CO₂ for 48 hrs. After 48 hrs the cells are harvested and RNA extraction is performed using the miRNeasy kit (Qiagen) following manufactures instructions, cDNA is synthesized using the reverse transcription kit (Applied Biosystems) and real time PCR is done to measure the transcription levels of miR130b target genes Glucocorticoid receptor, GR-α. MM.Re cell line is treated with 30 nM miR130b/miR130b*Cy3 AuNPs and the gene expression of miR130b target gene GR-α is analyzed by qPCR after 48 hrs. A 40% reduction in the expression of the target gene is observed upon miR130b/miR130b*Cy3 AuNPs treatment. Asterisk designates statistical significance.

### Intracellular Uptake and Target Downregulation of AuNP-miRNA/miRNA*Cy3 or IFHD-AuNP-miRNA/miRNA*Cy3

To assay the intracellular uptake of AuNP-miRNA/miRNA*Cy3 or IFHD-AuNP cell lines are seeded in 24 wells plates and treated with a suitable concentration of AuNP-miRNA/miRNA*Cy3 or IFHD-AuNP. Treatments are performed as follows: AuNP-miRNA/miRNA*Cy3 or IFHD-AuNP are resuspended in OptiMeM media (GIBCO) supplemented with 5% of Fetal Bovine Serum (FBS) and adequate volume is added directly to the cells on the well to reach the desire final concentration, cells are incubated at 37° C. in 5% CO₂; After 20 min. a small aliquot is taken and used to performed live-cell imaging using confocal microscopy. Cells are loaded in a neubauer hemocytometer chamber and the number of total cells and Cy3 positive cells are counted. The rest of the cells are incubated at 37° C. in 5% CO₂ for 48 hrs. After 48 hrs the cells are harvested and RNA extraction is performed using the miRNeasy kit (Qiagen) following manufactures instructions, cDNA is synthesized using the reverse transcription kit (Applied Biosystems) and real time PCR is done to measure the transcription levels of miR target genes. This protocol can be carried out also for Cy5-miRNA duplexes-conjugated AuNPs or IFHD-AuNPs.

The following references are cited herein.
1. Frens et al., Nature, Phys. Sci., 1973, 241, 20-22.
2. Giljohann et al. J. Am. Chem. Soc. 2009, 131:2072.
3. Patil et al. Bioconjugate Chem. 2008, 19:1396.
4. Lee et al. Bioconjugate Chem. 2004, 14:214.
5. Lee et al. Biochemical and Biophysical Research Communication 2007, 357:511.
6. Rosenberg et al. Nature, 1969, 222:385.
7. Dhar et al. J. Am. Chem. Soc. 2009, 131:14652.
8. Bernhardt et al. Inorganica Chimica Acta, 2004, 357, 4452.
9. Sideratou et al. Gene. Ther. Mol. Biol. 2006, 10:71.

Any patents or publications mentioned in this specification are indicative of the levels of those skilled in the art to which the invention pertains. These patents and publications are herein incorporated by reference to the same extent as if each individual publication was incorporated specifically and individually by reference.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. A nanoparticle platform, comprising;
a gold nanoparticle; and
a plurality of thiloated hyperbranched dendrons conjugated to the nanoparticle surface, said hyperbranched dendrons comprising chemically-modifiable surface groups, functionalized interior groups and nano-cavities within the hyperbranched structure.

2. The nanoparticle platform of claim 1, further comprising:
one or more of thiolated oligoethylene glycol, thiolated polyethylene glycol linkers or thiolated dendrons conjugated to uncovered nanoparticle surface areas or one or both of thiolated oligoethylene glycol or thiolated polyethylene glycol linkers conjugated to thiolated dendrons.

3. The nanoparticle platform of claim 1, further comprising one or more payload molecules conjugated to the interior or surface groups or within the nano-cavities or a combination thereof.

4. The nanoparticle platform of claim 1, wherein the payload molecules are one or more therapeutic agents or a non-cytotoxic signaling molecule or a combination thereof.

5. The nanoparticle platform of claim 4, wherein the therapeutic agent comprises one or more nucleic acids, one or more anticancer drugs, one or more small molecule inhibitors, or a combination thereof.

6. The nanoparticle platform of claim 5, wherein the nucleic acid is one or more of a microRNA, a small-interfering RNA or a DNA

7. The nanoparticle platform of claim 5, wherein the nucleic acid further comprises a thiolated polyethylene glycol linker or a thiolated oligoethylene glycol linker conjugated to an antisense strand of the nucleic acid.

8. The nanoparticle platform of claim 5, wherein the therapeutic agent is one or more nucleic acids, said nucleic acids electrostatically linked to the functionalized interior groups.

9. The nanoparticle platform of claim 5. wherein the therapeutic agent is one or more anticancer drugs or one or more small molecule inhibitors or a combination thereof, said agent(s) conjugated to chemically-modified surface groups or said agent(s) comprise a chemical modification and are conjugated directly to unmodified surface groups.

10. The nanoparticle platform of claim 5, wherein the anticancer drug is cisplatin.

11. The nanoparticle platform of claim 5, wherein the small molecule inhibitor is MK-1775.

12. The nanoparticle platform of claim 4, wherein the non-cytotoxic signaling agent is fluorescein isothiocyanate.

13. The nanoparticle platform of claim 1, wherein the surface groups comprise tri(hydroxymethyl)amidomethane and the interior groups comprise a tertiary amine.

14. The nanoparticle platform of claim 1, wherein the surface groups are chemically modified with an amino, sodium carboxylate, amido ethanol, succinamic acid, hexylamine, or amidoethylethanolamine moiety.

15. A combinatorial drug delivery vehicle, comprising:
a plurality of the nanoparticle platforms of claim 1; and
two or more different therapeutic agents conjugated to the surface and interior groups of the thiolated hyperbranched dendrons comprising the nanoparticle platforms.

16. The combinatorial drug delivery vehicle of claim 15, further comprising a non-cytotoxic signaling agent conjugated to the surface groups.

17. The combinatorial drug delivery vehicle of claim 16, wherein the non-cytotoxic signaling molecule is fluorescein isothiocyanate.

18. A method for delivering one or more therapeutic agents to a cell or tissue, comprising:
contacting the cell with the drug delivery vehicle of claim 1, said payload molecules comprising therapeutic agents, such that the drug delivery vehicle is internalized into the cell or tissue, thereby delivering the one or more therapeutic agents thereto.

19. The method of claim 18, wherein the payload molecules further comprise a non-cytotoxic signaling agent, the method further comprising:
monitoring a signal from the signaling agent, thereby detecting the drug delivery vehicle in the cell.

20. The method of claim 19, wherein the non-cytotoxic signaling agent is fluorescein isothiocyanate.

21. The method of claim 18, wherein the therapeutic agents are a microRNA, cisplatin and MK-1775.

22. The method of claim 18, wherein the contacting step occurs in vitro or in vivo.

23. A method for treating a pathophysiological condition in a subject, comprising:
administering, to the subject, an amount of the combinatorial drug delivery vehicle of claim 15 effective to deliver a pharmacological amount of payload molecules to cells or tissues associated with the pathophysiological condition, said payload molecules comprising therapeutic agents, thereby treating the pathophysiological condition.

24. The method of claim 23, wherein the therapeutic agents are a microRNA, cisplatin and MK-1775.

25. The method of claim 23, wherein the pathophysiological condition is a cancer.

26. A combinatorial drug delivery vehicle, comprising:
a plurality of gold nanoparticles;
a plurality of thilolated hyperbranched dendrons conjugated to the nanoparticle surfaces, said hyperbranched dendrons comprising chemically-modifiable tri(hydroxymethyl)amidomethane surface groups, tertiary amine interior groups and nano-cavities within the hyperbranched structure;
anticancer drugs and small molecule inhibitors individually conjugated to the tri(hydroxymethyl)amidomethane surface groups, either said surface group or said anticancer drugs and small molecule inhibitors further comprising a chemical modifier; and
microRNA duplexes electrostatically linked to the tertiary amine comprising the interior groups.

27. The combinatorial drug delivery vehicle of claim 26, further comprising fluorescein isothiocyanate conjugated to the tri(hydroxymethyl)amidomethane surface groups.

28. The combinatorial drug delivery vehicle of claim 26, wherein one or both of the unconjugated tri(hydroxymethyl)amidomethane surface groups or an antisense strand of the microRNA duplex further comprises a thiolated polyethylene glycol linker or a thiolated oligoethylene glycol linker.

29. The combinatorial drug delivery vehicle of claim 26, wherein the chemical modifier is an amino, sodium carboxylate, amido ethanol, succinamic acid, hexylamide, or amidoethylethanolamine moiety.

30. The combinatorial drug delivery vehicle of claim 26, wherein the anticancer drug is cisplating and the small molecule inhibitor is MK-1775.
